(19) United States
(12) Patent Application Publication
(10) Pub. No.: US 2012/0178754 A1
(43) Pub. Date: Jul. 12, 2012

(54) Furazano '3, 4-B! Pyrazines and Their Use as Anti-Tumor Agents

(75) Inventors: Paul W. Baures, Brunswick, OK (US); Donald R. James, El Sobrante, CA (US); Richard D. Gless, Oakland, CA (US); Thuy Tran, San Jose, CA (US); Herman J. Verheij, Papendrecht (NL); Jan C.C. Schultz, The Hague (NL)

(73) Assignee: Compass Pharmaceuticals LLC, Union, NJ (US)

(21) Appl. No.: 13/427,012

(22) Filed: Mar. 22, 2012

Related U.S. Application Data
(63) Continuation of application No. 12/777,018, filed on May 10, 2010, now abandoned.
Application No. 12/777,018 is a division of application No. 11/664,241, filed on Feb. 4, 2009, now abandoned.

(30) Foreign Application Priority Data
Oct. 12, 2005 (US) PCT/US05/36554

(51) Int. Cl. A61K 31/4985; A61K 31/5377; A61P 35/04

(52) U.S. Cl. 514/234.2; 514/249

(57) ABSTRACT

The present invention provides novel compounds and pharmaceutical compositions thereof, as well as methods for using the compounds and pharmaceutical compositions for treating tumors. Examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

### CROSS REFERENCE

This application is a continuation of U.S. patent application Ser. No. 12/777,018, filed May 10, 2010, which is a divisional of U.S. patent application Ser. No. 11/664241, filed Feb. 4, 2009, which claims priority to U.S. provisional patent application Ser. No. 60/618,800 filed Oct. 14, 2004, both incorporated by reference herein in their entirety.

### BACKGROUND OF THE INVENTION

Approximately twenty percent of deaths from all causes in the United States are cancer-related. Although chemotherapy is a principal means of cancer treatment, the rate at which effective new drugs have become available for use in cancer chemotherapy has not increased (Horowitz et al., Journal of Clinical Oncology, Vol. 6, No. 2, pp. 308-314 (1988)). Despite many years of promising new therapies, cancer remains a major cause of morbidity and mortality (Bailar et al., N. Engl. J. Med. 336:1569-1574, 1997). Accordingly, there is a substantial need for new drugs that are effective in inhibiting the growth of tumors.

### SUMMARY OF THE INVENTION

The present invention provides novel compounds and pharmaceutical compositions thereof, as well as methods for using the compounds and pharmaceutical compositions for treating tumors. Examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

In one aspect, the present invention provides novel compounds according the general formula I:

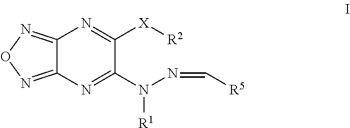

wherein

### X is O, S, or NR;

R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN;

R and R¹ independently are hydrogen or lower alkyl;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
   R⁷ and R⁸ are independently hydrogen, lower alkyl, aryl, heteroaryl, arylalkyl, heteroarylalkyl, —C(O)R¹⁰ or —C(O)OR′;
   R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
   R′ is hydrogen or lower alkyl;
   and pharmaceutically acceptable derivatives thereof

In another aspect the present invention provides pharmaceutical compositions, comprising one or more compounds according to the invention, and a pharmaceutically acceptable carrier.

In another aspect, the present invention provides methods for treating a subject with a tumor, comprising administering to the subject an amount effective of a compound according to formula II:

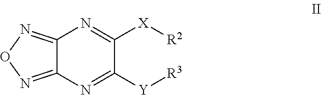

wherein

### X is O, S, or NR;

### Y is O, S, or NR¹;

R and R¹ are independently hydrogen or lower alkyl;
R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C6)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN, or
R² is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰,or
R² is heteroarylalkyl, or
R and R², when Y is —NR¹, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R³ is hydrogen, or
R³ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, —C(O)OR′, —C(O)NR⁷R⁸, —C(O)R¹⁰ and —CN, or

### R³ is —N═CH—R⁵, or

R³ is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰, or
R¹ and R³, when Y is —NR¹, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R² and R³, together with the heteroatom that they are attached, form a 5-20 membered heterocyclic ring optionally substituted with one or two groups selected from lower alkyl, —C(O)R¹⁰, —C(O)OR′, —OC(O)R′, —C(O)NR⁷R⁸, oxo, aryl, halo, hydroxyl, trifluoromethyl, —NR⁷R⁸ and lower alkoxy;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
   R⁶ is lower alkyl optionally substituted with one or two groups selected from hydroxyl and lower alkoxy,
   R⁷ and R⁸ are independently hydrogen, lower alkyl, —C(O)R¹⁰ or —C(O)OR′, or
   R⁷ and R⁸ are independently aryl, heteroaryl, arylalkyl, heteroarylalkyl, each of which may be optionally substituted with one or two groups selected from lower alkyl, lower alkoxy, oxo, hydroxyl or amino;
   R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
   R′ is hydrogen or lower alkyl; and pharmaceutically acceptable derivatives thereof.

### BRIEF DESCRIPTION OF THE FIGURES

FIG. 1 is a table of data for representative compounds of the invention.

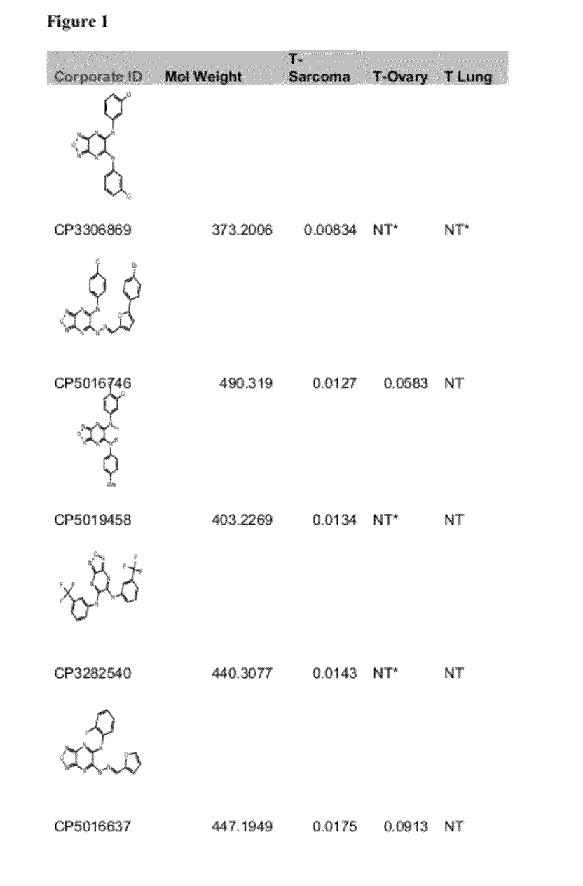

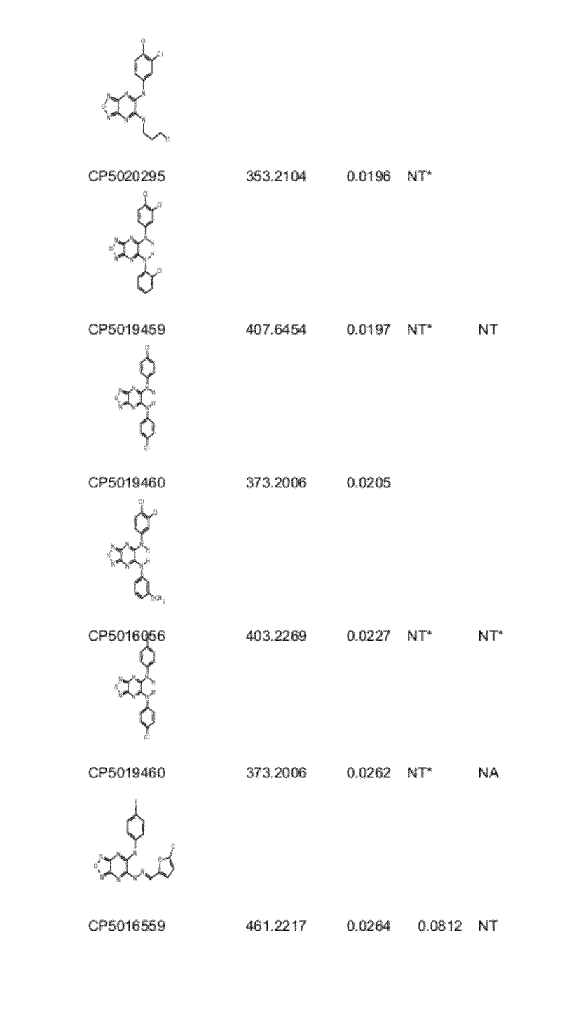

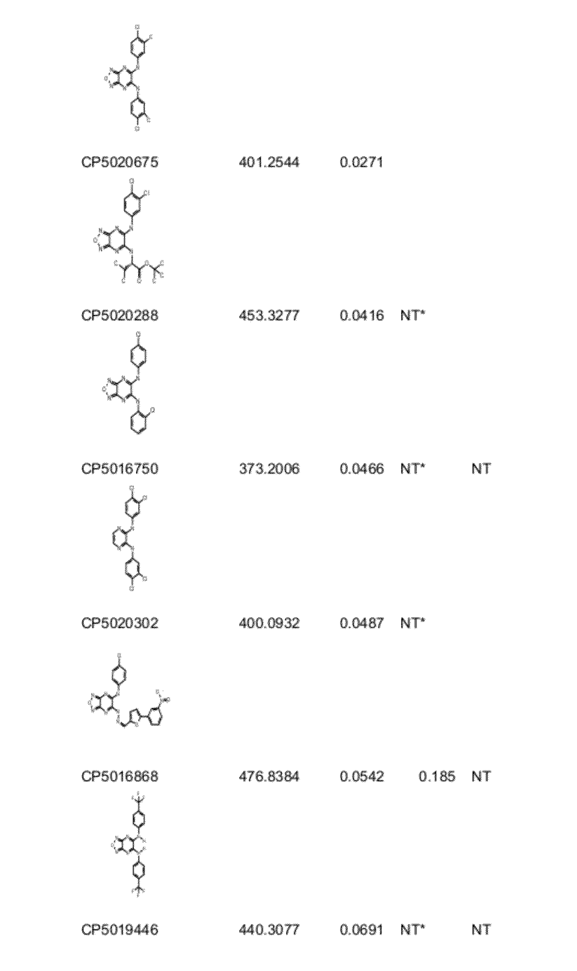

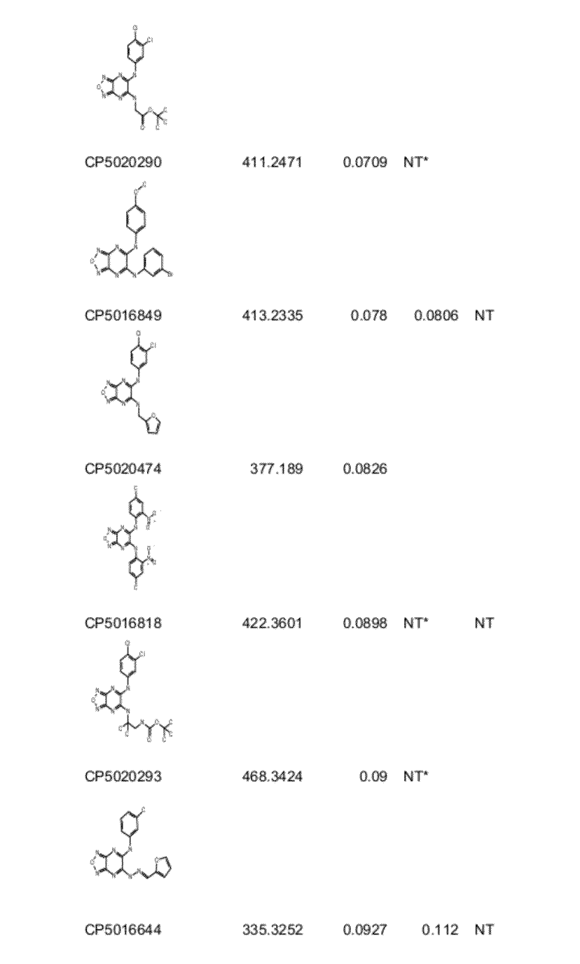

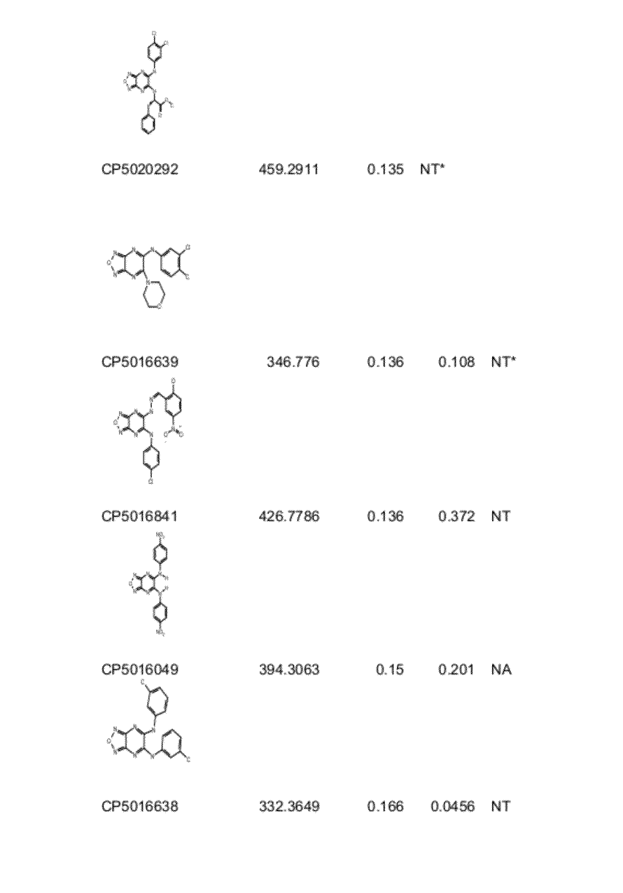

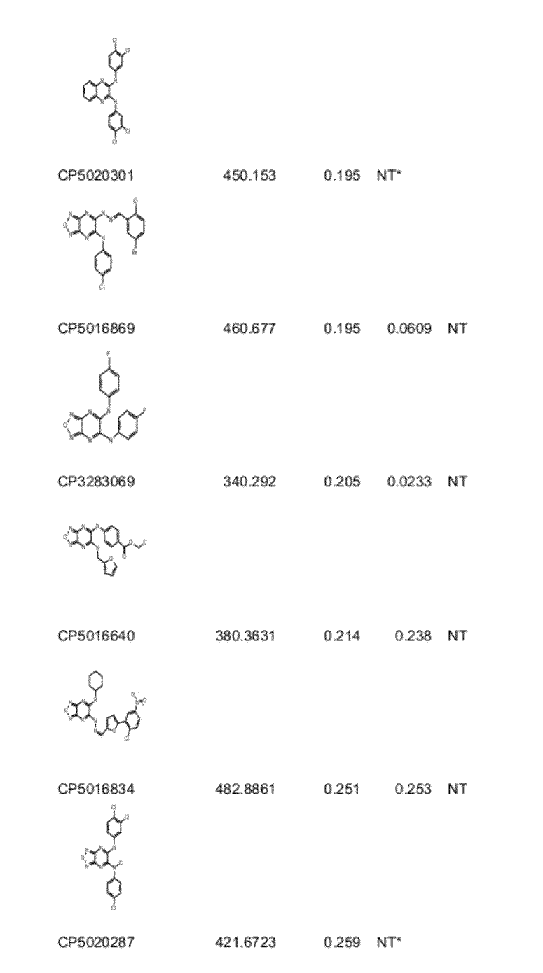

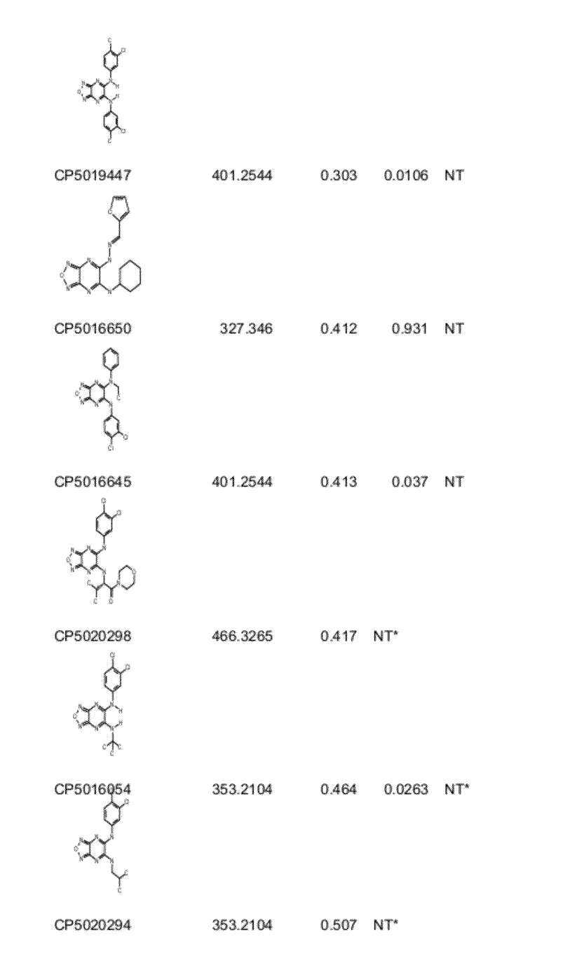

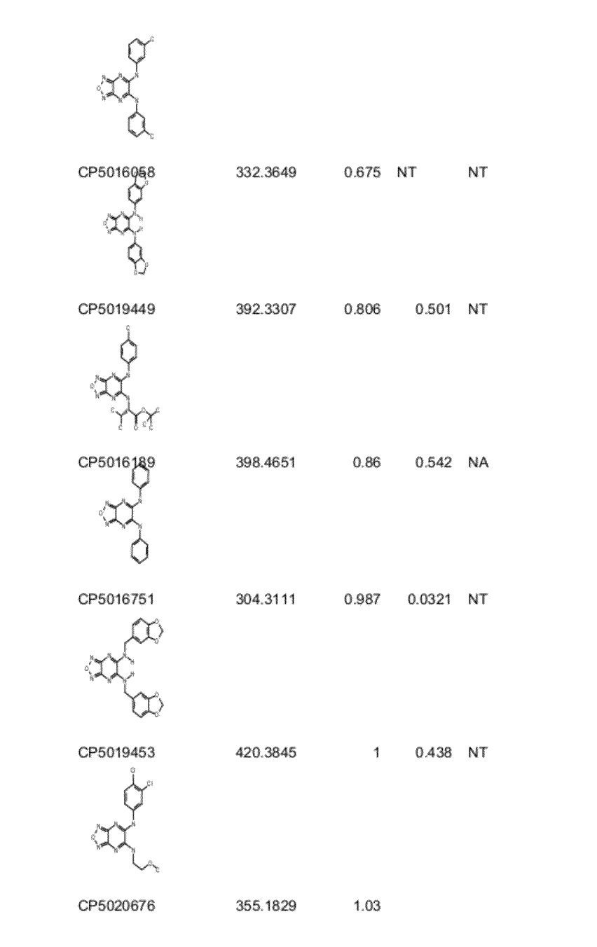

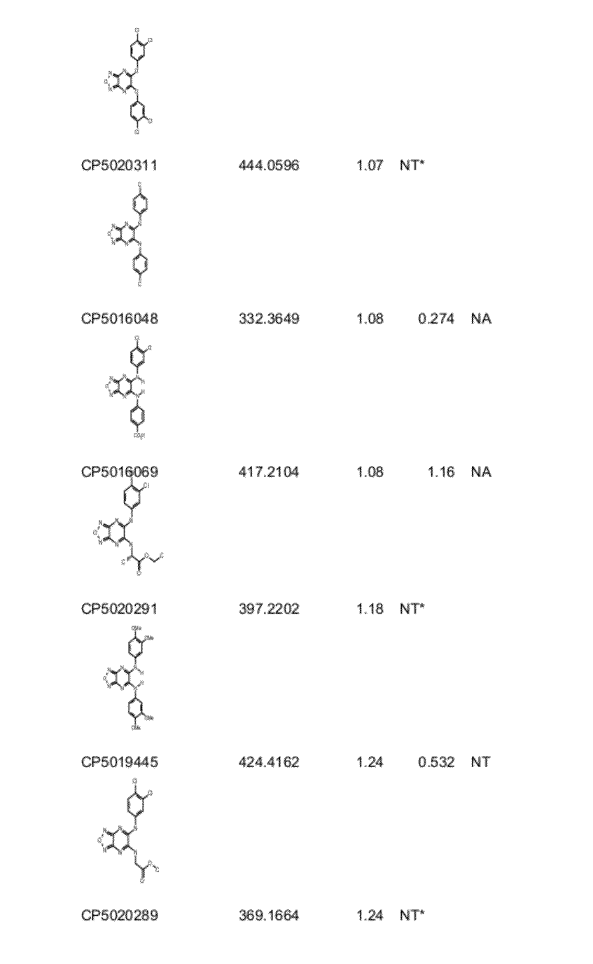

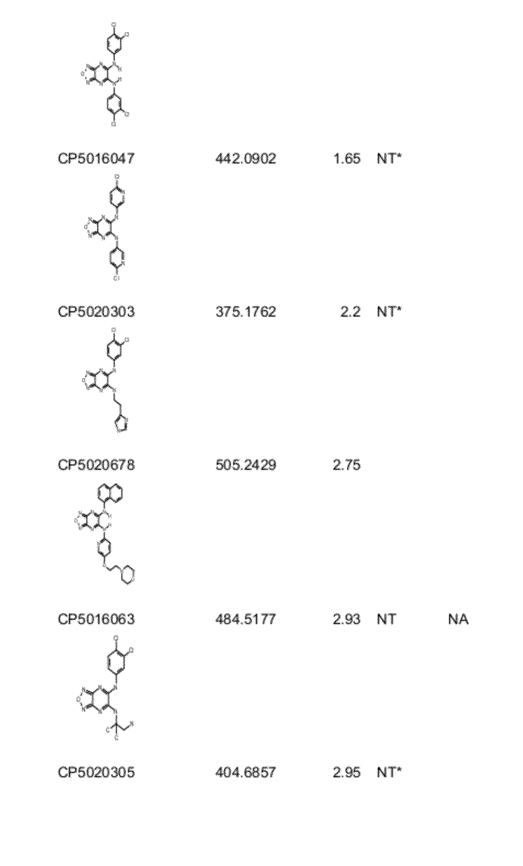

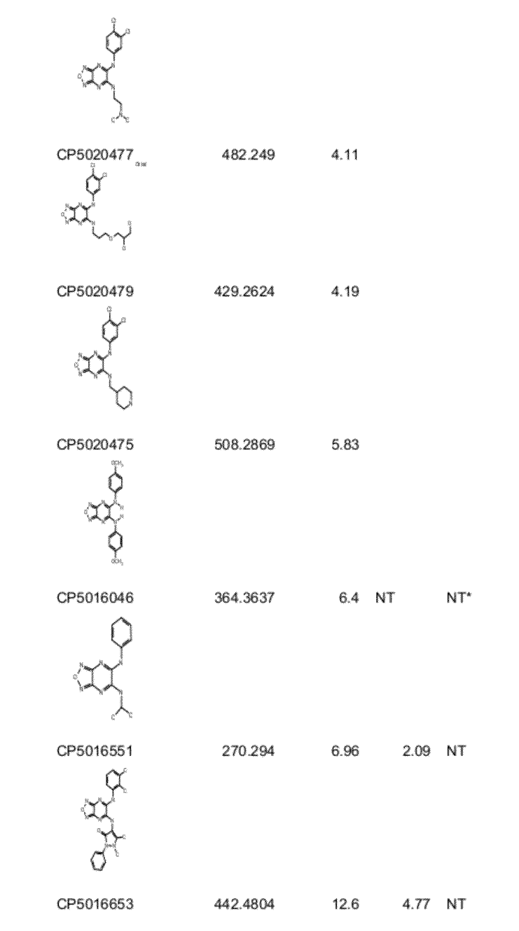

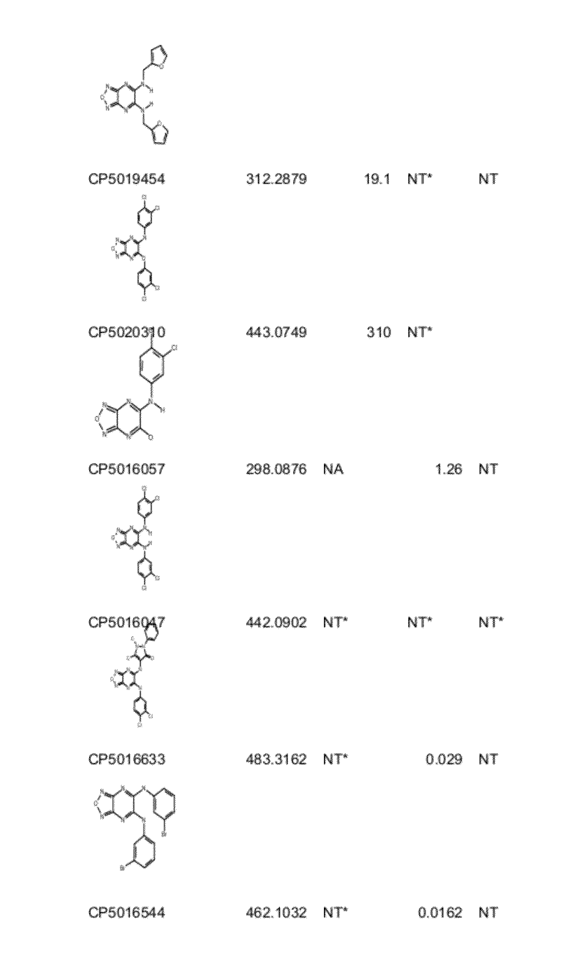

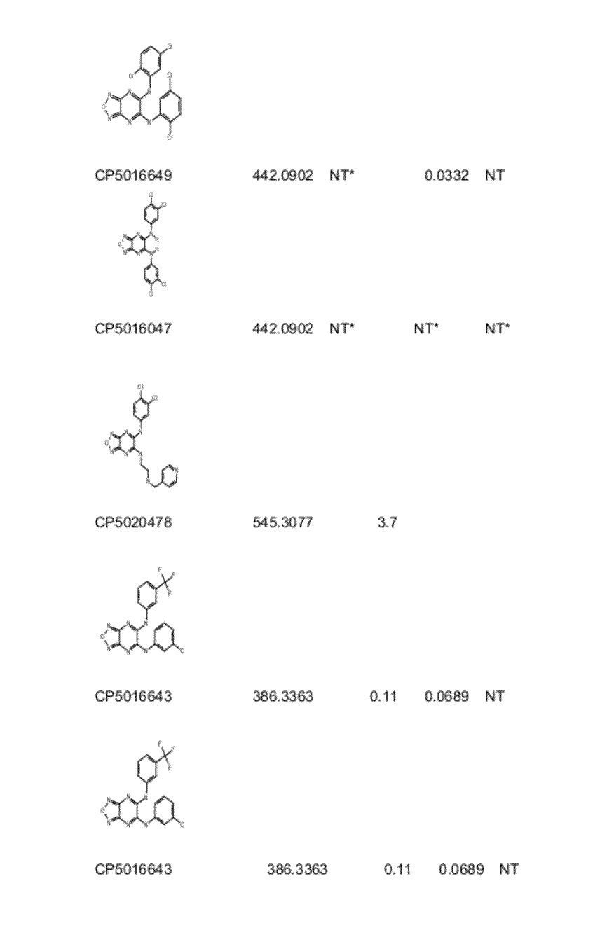

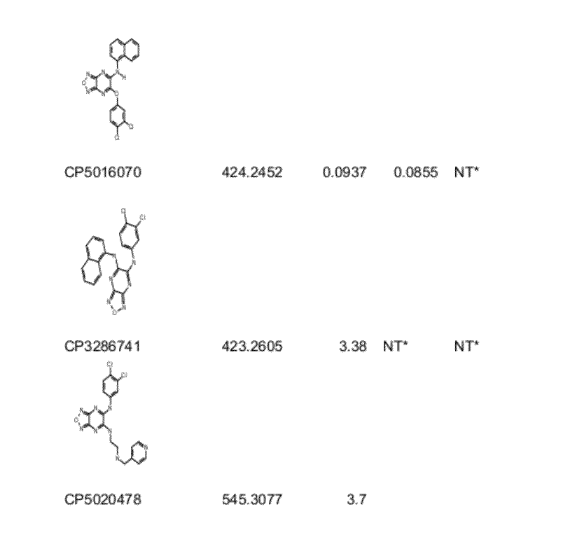

### DETAILED DESCRIPTION OF THE INVENTION

All references cited herein are incorporated by reference in their entirety. In one aspect, the present invention provides novel compounds according the general formula I:

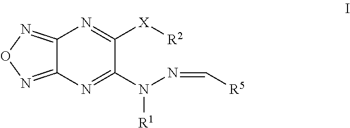

wherein

### X is O, S, or NR;

R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN;
R and R¹ independently are hydrogen or lower alkyl;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
   R⁷ and R⁸ are independently hydrogen, lower alkyl, aryl, heteroaryl, arylalkyl, heteroarylalkyl, —C(O)R¹⁰ or —C(O)OR′;
   R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
   R′ is hydrogen or lower alkyl;
   and pharmaceutically acceptable derivatives thereof.

The invention also relates to compounds of formula I wherein the substituted hydrazone is a cis geometric isomer.

The invention also relates to compounds of formula I wherein the substituted hydrazone is a trans geometric isomer. In an embodiment, the invention relates to compounds of formula I wherein R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

In another embodiment, the invention relates to compounds of formula I wherein R and R¹ are hydrogen.

In yet another embodiment, the invention relates to compounds of formula I wherein R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

In still another embodiment, the invention relates to compounds of formula I wherein X is —NR—.

The invention also relates to compounds of formula Ia:

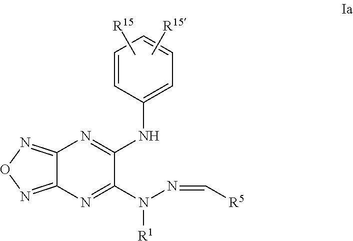

wherein R¹ and R⁵ as are defined above for formula I and R¹⁵ and R^{15′} are independently hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′, —C(O)NR⁷R⁸, —C(O)R¹⁰ and —CN.

The invention also relates to compounds of formula Ia wherein the substituted hydrazone is a cis geometric isomer.

The invention also relates to compounds of formula Ia wherein the substituted hydrazone is a trans geometric isomer.

In an embodiment, the invention relates to compounds of formula Ia wherein R¹ is hydrogen.

In still another embodiment, the invention relates to compounds of formula Ia wherein R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

In yet another embodiment, the invention relates to compounds of formula Ia wherein R¹⁵ and R¹⁵ are independently hydrogen, trifluoromethyl, nitro, lower alkoxy, halo or lower alkyl.

The invention also relates to compounds of formula Ib:

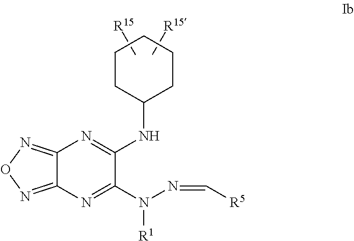

wherein R¹ and R⁵ as are defined above for formula I and R¹⁵ and R^{15′} are as defined above for formula Ia.

The invention also relates to compounds of formula Ib wherein the substituted hydrazone is a cis geometric isomer.

The invention also relates to compounds of formula Ib wherein the substituted hydrazone is a trans geometric isomer.

In an embodiment, the invention relates to compounds of formula Ib wherein R¹ is hydrogen.

In another embodiment, the invention relates to compounds of formula Ib wherein R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

In yet another embodiment, the invention relates to compounds of formula Ib wherein R¹⁵ and R¹⁵ are independently hydrogen, trifluoromethyl, nitro, lower alkoxy, halo or lower alkyl.

The invention also relates to compounds of formula Ic:

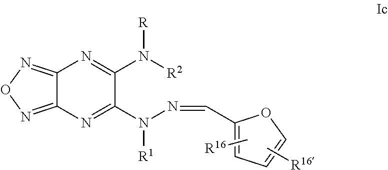

wherein R, R¹ and R² as are defined above for formula I and R¹⁶ and R^{16′} independently are hydrogen, lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

The invention also relates to compounds of formula Ic wherein the substituted hydrazone is a cis geometric isomer.

The invention also relates to compounds of formula Ic wherein the substituted hydrazone is a trans geometric isomer.

In an embodiment, the invention relates to compounds of formula Ic wherein R and R¹ are hydrogen.

In another embodiment, the invention relates to compounds of formula Ic wherein R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

In yet another embodiment, the invention relates to compounds of formula Ic wherein R¹⁶ and R^{16′} independently are hydrogen, lower alkyl, lower alkoxy, nitro, halo, or aryl optionally substituted with one or two groups independently selected from lower alkyl, lower alkoxy, hydroxyl, trifluoromethyl, halo, —C(O)OR′, —C(O)NR⁷R⁸, —C(O)R¹⁰ , nitro and —CN.

The invention also relates to compounds of formula Id:

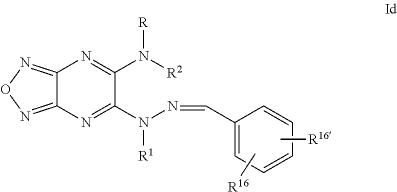

wherein R, R¹ and R² as are defined above for formula I and R¹⁶ and R^{16′} are as defined above for formula Ic.

The invention also relates to compounds of formula Id wherein the substituted hydrazone is a cis geometric isomer.

The invention also relates to compounds of formula Id wherein the substituted hydrazone is a trans geometric isomer.

In an embodiment, the invention relates to compounds of formula Id wherein R and R¹ are hydrogen.

In still another embodiment, the invention relates to compounds of formula Id wherein R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

In yet another embodiment, the invention relates to compounds of formula Id wherein R¹⁶ and R^{16′} independently are hydrogen, lower alkyl, lower alkoxy, nitro, halo, or aryl optionally substituted with one or two groups independently selected from lower alkyl, lower alkoxy, hydroxyl, trifluoromethyl, halo, —C(O)OR′, —C(O)NR⁷R⁸, —C(O)R¹⁰ , nitro and —CN.

A family of specific compounds of particular interest within formula I consists of compounds and pharmaceutically-acceptable salts thereof as follows (all compounds are named via the structure naming plug-in to either ChemDraw Ultra 6.0 or 8.0):

(a) 6-(2-((furan-2-yl)methylene)hydrazinyl)-N-(2-iodophenyl)-[1,2,5]oxadiazolo[3,4-b]pyrazin-5-amine ;

(b) 6-(2-((5-methylfuran-2-yl)methylene)hydrazinyl)-N-(4-iodophenyl)-[1,2,5]oxadiazolo[3,4-b]pyrazin-5-amine;

(c) 6-(2-45-(3-nitrophenyl)furan-2-yl)methylene)hydrazinyl)-N-(4-chloro-2-iodophenyl)-[1,2,5]oxadiazolo [3,4-b]pyrazin-5-amine;

(d) 6-(2-((furan-2-yl)methylene)hydrazinyl)-N-m-tolyl-[1,2,5]oxadiazolo[3,4-b]pyrazin-5-amine;

(e) 6-(2-((2-hydroxy-5-nitro-phenyl-1-yl)methylene)hydrazinyl)-N-(4-chloro-phenyl) [1,2,5]oxadiazolo[3,4-b]pyrazin-5-amine;

(f) 6-(2-((2-hydroxy-5-bromo-phenyl)methylene)hydrazinyl)-N-(4-chloro-phenyl) [1,2,5]oxadiazolo[3,4-b]pyrazin-5-amine;

(g) 6-(2-45-(2-chloro-5-nitrophenyl)furan-2-yl)methylene)hydrazinyl)-N—cyclohexyl-[1,2,5]oxadiazolo[3,4-b]pyrazin-5-amine;

(h) 6-(2-((furan-2-yl)methylene)hydrazinyl)-N—cyclohexyl-[1,2,5]oxadiazolo[3,4-b]pyrazin-5-amine;

(i) 6-(2-45-(2-methoxy-4-nitrophenyl)furan-2-yl)methylene)hydrazinyl)-N-(4-chlorophenyl)-[1,2,5]oxadiazolo[3,4-b]pyrazin-5-amine;

(j) 64(E)-245-(4-bromophenyl)furan-2-yl)methylene)hydrazinyl)-N—p-tolyl-[1,2,5]oxadiazolo[3,4-b]pyrazin-5-amine; and

(k) 6-(2-((2-hydroxy-5-nitro-phenyl)methylene)hydrazinyl)-N-(2,3-dimethyl-phenyl)[1,2,5]oxadiazolo[3,4-b]pyrazin-5-amine.

The compounds of the invention include pharmaceutically acceptable salts, esters, amides, and prodrugs therof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977;66:1-19 which is incorporated herein by reference.)

Examples of pharmaceutically acceptable, non-toxic esters of the compounds of this invention include C₁-C₆ alkyl esters, wherein the alkyl group is a straight or branched, substituted or unsubstituted, C₅-C₇ cycloalkyl esters, as well as arylalkyl esters such as benzyl and triphenylmethyl. C₁-C₄ alkyl esters are preferred, such as methyl, ethyl, 2,2,2-trichloroethyl, and tert-butyl. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods. The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

These compounds can be administered individually or in combination, usually in the form of a pharmaceutical composition. Such compositions are prepared in a manner well known in the pharmaceutical art and comprise at least one active compound. Accordingly, a further aspect of the present invention includes pharmaceutical compositions comprising as one or more compounds of the invention disclosed above, associated with a pharmaceutically acceptable carrier. For administration, the compounds are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In another aspect, the present invention provides methods for treating a subject with a tumor, comprising administering to the subject an amount effective of a compound according to formula II:

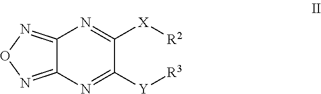

wherein

### X is O, S, or NR;

### Y is O, S, or NR¹;

R and R¹ are independently hydrogen or lower alkyl;
R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN, or
R² is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰,or
R² is heteroarylalkyl, or
R and R², when Y is —NR¹, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R³ is hydrogen, or
R³ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C6)alkoxy(C₁-C₆)alkyl, —NR⁷R⁸, nitro, oxo, aryl, —C(O)OR′, —C(O)NR⁷R⁸, —C(O)R¹⁰ and —CN, or

### R³ is —N═CH—R⁵, or

R³ is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰, or
R¹ and R³, when Y is —NR^{I}, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R² and R³, together with the heteroatom that they are attached, form a 5-20 membered heterocyclic ring optionally substituted with one or two groups selected from lower alkyl, —C(O)R¹⁰, —C(O)OR′, —OC(O)R′, —C(O)NR⁷R⁸, oxo, aryl, halo, hydroxyl, trifluoromethyl, —NR⁷R⁸ and lower alkoxy;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
   R⁶ is lower alkyl optionally substituted with one or two groups selected from hydroxyl and lower alkoxy,
   R⁷ and R⁸ are independently hydrogen, lower alkyl, —C(O)R¹⁰ or —C(O)OR′, or
   R⁷ and R⁸ are independently aryl, heteroaryl, arylalkyl, heteroarylalkyl, each of which may be optionally substituted with one or two groups selected from lower alkyl, lower alkoxy, oxo, hydroxyl or amino;
   R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
   R′ is hydrogen or lower alkyl; and pharmaceutically acceptable derivatives thereof.

The invention also relates to methods for treating a subject with a tumor, comprising administering to the subject an amount effective of a compound according to formula IIa:

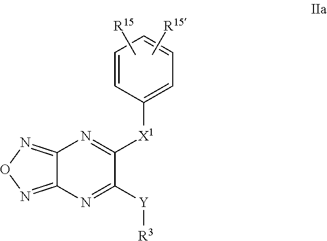

wherein Y and R³ are as defined above for formula II, R¹⁵ and R^{15′} are as defined above for formula Ia, X¹ is O or —NR—, and R is as defined above for formula II.

The invention also relates to methods for treating a subject with a tumor, comprising administering to the subject an amount effective of a compound according to formula IIb:

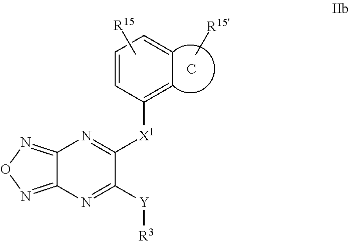

wherein Y and R³ are as defined above for formula II, R¹⁵ and R^{15′} are as defined above for formula Ia, X¹ is as defined above for formula IIa, R is as defined above for formula II, and the c-ring, together with the phenyl ring to which it is attached, is a 9-11 membered aryl, heteroaryl, cycloalkyl or heterocyclyl group.

The invention also relates to methods for treating a subject with a tumor, comprising administering to the subject an amount effective of a compound according to formula IIc:

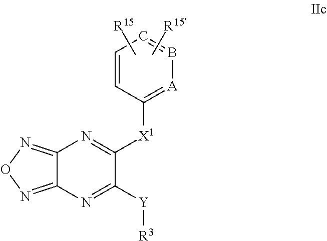

wherein Y and R³ are as defined above for formula II, R¹⁵ and R^{15′} are as defined above for formula Ia, X¹ is as defined above for formula Ia, R is as defined above for formula II, and one of A, B and C is N.

The invention also relates to methods for treating a subject with a tumor, comprising administering to the subject an amount effective of a compound according to formula IId:

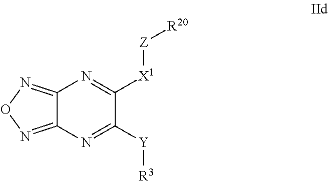

wherein Y and R³ are as defined above for formula II, X¹ is as defined above for formula Ia, Z is lower alkyl optionally substituted with one or two groups selected from hydroxyl, lower alkoxy or halo, and R²⁰ is nothing or —C(O)OR′, —NC(O)R′, —NR⁷R⁸, aryl or heteroaryl, wherein R′, R⁷ and R⁸ are as defined above for formula II.

The invention also relates to methods for treating a subject with a tumor, comprising administering to the subject an amount effective of a compound according to formula IIe:

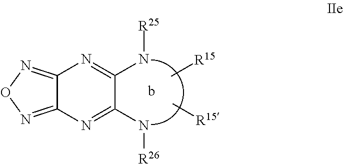

wherein R¹⁵ and R^{15′} are as defined above for formula Ia, R²⁵ and R²⁶ are independently hydrogen, aryl or lower alkyl, and the b-ring is a 5-20 membered heterocyclic ring.

The invention also relates to methods for treating a subject with a tumor, comprising administering to the subject an amount effective of a compound according to formula IIf:

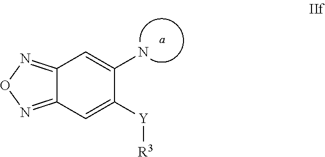

wherein Y and R³ are as defined above for formula II, and the a-ring, which includes the nitrogen atom, is a 5-7 membered heterocyclic ring optionally substituted with one or two groups that, independently, are R¹⁵ or R^{15′}, wherein R¹⁵ and R^{15′} are as defined above in formula Ia.

The invention also relates to methods for treating a subject with a tumor, comprising administering to the subject an amount effective of a compound according to formula Hg:

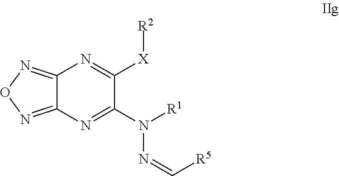

wherein X, R¹, R² and R⁵ are as defined above for formula I.

The invention also relates to methods for treating a subject with a tumor, comprising administering to the subject an amount effective of a compound according to formula IIh:

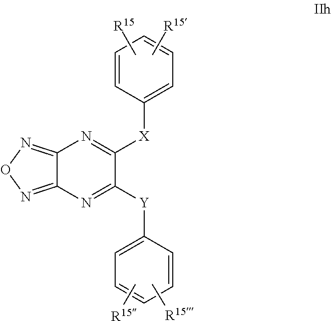

wherein X, Y, R¹⁵ and R^{15′} are as defined above, and R^{15″} and R^{15′″} are as defined above for R¹⁵ and R^{15′}.

The invention also relates to methods for treating a subject with a tumor, comprising administering to the subject an amount effective of a compound according to formula IIi:

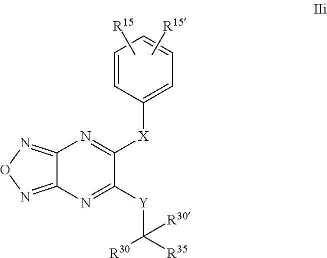

wherein X, Y, R¹⁵ and R^{15′} are as defined above, R³⁰ and R^{30′} are independently hydrogen, arylalkyl or lower alkyl, and R³⁵ is hydrogen, lower alkyl, lower alkoxy, —C(O)OR′, —C(O)R¹⁰, —NR⁷R⁸, aryl, heteroaryl, cycloalkyl, or heterocycloalkyl.

The invention also relates to the following compounds and to methods for treating a subject with a tumor by administering to a subject the following compounds (all compounds are named via the structure naming plug-in to either ChemDraw Ultra 6.0 or 8.0):
(a) N5,N6-bis(3-chlorophenyl)-[1,2,5]oxadiazolo[3,4-b]pyrazine-5,6-diamine;
(b) N5-(3,4-dichlorophenyl)-N6-(4-methoxyphenyl)-[1,2,5]oxadiazolo[3,4-b]pyrazine-5,6-diamine;
(c) N5,N6-bis(3-trifluoromethyl)-[1,2,5]oxadiazolo[3,4-b]pyrazine-5,6-diamine;
(d) N5-butyl-N6-(3,4-dichlorophenyl)-[1,2,5]oxadiazolo[3,4-b]pyrazine-5,6-diamine;
(e) N5-(3,4-dichlorophenyl)-N6-(2-chlorophenyl)-[1,2,5]oxadiazolo[3,4-b]pyrazine-5,6-diamine;
(f) N5,N6-bis(4-chlorophenyl)-[1,2,5]oxadiazolo[3,4-b]pyrazine-5,6-diamine;
(g) N5-(3,4-dichlorophenyl)-N6-(3-methoxyphenyl)-[1,2,5]oxadiazolo[3,4-b]pyrazine-5,6-diamine;
(h) N5,N6-bis(4-chloro-3-methylphenyl)-[1,2,5]oxadiazolo[3,4-b]pyrazine-5,6-diamine;
(i) (5)-tent butyl-2-(5-(4-chloro-3-methylphenylamino)-[1,2,5]oxadiazolo[3,4-b]pyrazin-6-ylamino)-3-methylbutanoate;
(j) N5-(4-chlorophenyl)-N6-(2-chlorophenyl)-[1,2,5]oxadiazolo[3,4-b]pyrazine-5,6-diamine;
(k) N5,N6-bis(3,4-dichlorophenyl)-[1,2,5]oxadiazolo[3,4-b]pyrazine-5,6-diamine;
(l) tert-butyl2-(5-(3,4-dichlorophenylamino)-[1,2,5]oxadiazolo[3,4-b]pyrazin-6-ylamino)acetate;
(m) N5-(4-methoxyphenyl)-N6-(3-bromophenyl)-[1,2,5]oxadiazolo[3,4-b]pyrazine-5,6-diamine;
(n) N5-(3,4-dichlorophenyl)-N6-((furan-2-yl)methyl)-[1,2,5]oxadiazolo[3,4-b]pyrazine-5,6-diamine;
(o) N5,N6-bis(4-trifluoromethylphenyl)-[1,2,5]oxadiazolo[3,4-b]pyrazine-5,6-diamine;
(p) N5,N6-bis(4-methyl-2-nitrophenyl)-[1,2,5]oxadiazolo[3,4-b]pyrazine-5,6-diamine;
(q) tert-butyl 2-(5-(3,4-dichlorophenylamino)-[1,2,5]oxadiazolo[3,4-b]pyrazin-6-ylamino)-2-methylpropylcarbamate;
(r) 6-(3,4-dichlorophenoxy)-N-(naphthalen-1-yl)-[1,2,5]oxadiazolo[3,4-b]pyrazin-5-amine;
(s) N5-(3-trifluormethylphenyl)-N6-(3-methylphenyl)-[1,2,5]oxadiazolo[3,4-b]pyrazine-5,6-diamine;
(t) (5)-methyl 2-(5-(3,4-dichlorophenylamino)-[1,2,5]oxadiazolo[3,4-b]pyrazin-6-ylamino)-3-phenylpropanoate;
(u) N-(3-chloro-4-methylphenyl)-6-morpholino-[1,2,5]oxadiazolo[3,4-b]pyrazin-5-amine;
(v) N5,N6-bis(4-nitrophenyl)-[1,2,5]oxadiazolo[3,4-b]pyrazine-5,6-diamine;
(w) N5,N6-bis(3-methylphenyl)-[1,2,5]oxadiazolo[3,4-b]pyrazine-5,6-diamine;
(x) N5,N6-bis(4-fluorophenyl)-[1,2,5]oxadiazolo[3,4-b]pyrazine-5,6-diamine;
(y) ethyl 4-(5-((furan-2-yl)methylamino)-[1,2,5]oxadiazolo[3,4-b]pyrazin-6-ylamino)benzoate;
(z) N6-(3,4-dichlorophenyl)-N5-(4-chlorophenyl)-N5-methyl-[1,2,5]oxadiazolo[3,4-b]pyrazine-5,6-diamine;
(aa) N5,N6-bis(3-chloro-4-methylphenyl)-[1,2,5]oxadiazolo[3,4-b]pyrazine-5,6-diamine;
(bb) N5-(3,4-dichlorophenyl)-N6-ethyl-N6-phenyl-[1,2,5]oxadiazolo[3,4-b]pyrazine-5,6-diamine;
(cc) (S)-2-(5-(3,4-dichlorophenylamino)-[1,2,5]oxadiazolo[3,4-b]pyrazin-6-ylamino)-3-methyl-1-morpholinobutan-1-one;
(dd) N5-tent-butyl-N6-(3,4-dichlorophenyl)-[1,2,5]oxadiazolo[3,4-b]pyrazine-5,6-diamine;
(ee) N5-(3,4-dichlorophenyl)-N6-isobutyl-[1,2,5]oxadiazolo[3,4-b]pyrazine-5,6-diamine;
(ff) N5,N6-bis(3-methylphenyl)-[1,2,5]oxadiazolo[3,4-b]pyrazine-5,6-diamine;
(gg) N5-(benzo [d][1,3]dioxo1-5-yl)-N6-(benzo [d][1,3]dioxo1-6-yl)-[1,2,5]oxadiazolo[3,4-b]pyrazine-5,6-diamine;
(hh) (S)-tent-butyl 2-(5-(p-tolylamino)-[1,2,5]oxadiazolo[3,4-b]pyrazin-6-ylamino)-3-methylbutanoate;
(ii) N5,N6-bis(phenyl)-[1,2,5]oxadiazolo[3,4-b]pyrazine-5,6-diamine;
(jj) N5-((benzo[d][1,3]dioxo1-5-yl)methyl)-N6-((benzo[d][1,3]dioxol-6-yl)methyl)-[1,2,5]oxadiazolo[3,4-b]pyrazine-5,6-diamine;
(kk) N5-(3,4-dichlorophenyl)-N6-(2-methoxyethyl)-[1,2,5]oxadiazolo[3,4-b]pyrazine-5,6-diamine;
(ll) 5,6-bis(3,4-dichlorophenoxy)-[1,2,5]oxadiazolo[3,4-b]pyrazine;
(mm) N5,N6-bis(4-methylphenyl)-[1,2,5]oxadiazolo[3,4-b]pyrazine-5,6-diamine;
(nn) 4-(5-(3,4-dichlorophenylamino)-[1,2,5]oxadiazolo[3,4-b]pyrazin-6-ylamino)benzoic acid;
(oo) (5)-ethyl2-(5-(3,4-dichlorophenylamino)-[1,2,5]oxadiazolo[3,4-b]pyrazin-6-ylamino)propanoate;
(pp) N5,N6-bis(3,4-dimethoxyphenyl)-[1,2,5]oxadiazolo[3,4-b]pyrazine-5,6-diamine;
(qq) methyl 2-(5-(3,4-dichlorophenylamino)-[1,2,5]oxadiazolo[3,4-b]pyrazin-6-ylamino)acetate;
(rr) N5,N6-bis(6-chloropyridin-3-yl)-[1,2,5]oxadiazolo[3,4-b]pyrazine-5,6-diamine;
(ss) N5-(2-(1H-imidazol-4-yl)ethyl)-N6-(3,4-dichlorophenyl)-[1,2,5]oxadiazolo[3,4-b]pyrazine-5,6-diamine;
(tt) N5-(5-(2-morpholinoethoxy)pyridin-2-yl)-N6-(naphthalen-1-yl)-[1,2,5]oxadiazolo[3,4-b]pyrazine-5,6-diamine;
(uu) N5-(1-amino-2-methylpropan-2-yl)-N6-(3,4-dichlorophenyl)-[1,2,5]oxadiazolo[3,4-b]pyrazine-5,6-diamine;
(vv) N5-(3,4-dichlorophenyl)-N6-(naphthalen-1-yl)-[1,2,5]oxadiazolo[3,4-b]pyrazine-5,6-diamine;
(ww) N5-(2-((pyridin-4-yl)methylamino)ethyl)-N6-(3,4-dichlorophenyl)-[1,2,5]oxadiazolo[3,4-b]pyrazine-5,6-diamine;
(xx) N5-(3,4-dichlorophenyl)-N6-(2-(dimethylamino)ethyl)-[1,2,5]oxadiazolo[3,4-b]pyrazine-5,6-diamine;
(yy) 3-(3-(5-(3,4-dichlorophenylamino)-[1,2,5]oxadiazolo[3,4-b]pyrazin-6-ylamino) propoxy)propane-1,2-diol;
(zz) N5-(3,4-dichlorophenyl)-N6-((piperidin-4-yl)methyl)-[1,2,5]oxadiazolo[3,4-b]pyrazine-5,6-diamine;
(aaa) N5,N6-bis(4-methoxyphenyl)-[1,2,5]oxadiazolo[3,4-b]pyrazine-5,6-diamine;
(bbb) N5-isopropyl-N6-phenyl-[1,2,5]oxadiazolo[3,4-b]pyrazine-5,6-diamine;
(ccc) 4-(5-(2,3-dimethylphenylamino)-[1,2,5]oxadiazolo[3,4-b]pyrazin-6-ylamino)-1,2-dihydro-2,3-dimethyl-1-phenylpyrazol-5-one;
(ddd) N5,N6-bis((furan-2-yl)methyl)[1,2,5]oxadiazolo[3,4-b]pyrazine-5,6-diamine;
(eee) 6-(3,4-dichlorophenoxy)-N-(3,4-dichlorophenyl)-[1,2,5]oxadiazolo[3,4-b]pyrazin-5-amine;
(fff) 6-(3,4-dichlorophenylamino)-[1,2,5]oxadiazolo[3,4-b]pyrazin-5-ol;
(ggg) 4-(5-(3,4-dichlorophenylamino)-[1,2,5]oxadiazolo[3,4-b]pyrazin-6-ylamino)-1,2-dihydro-2,3-dimethyl-1-phenylpyrazol-5-one;
(hhh) N5,N6-bis(3-bromophenyl)-[1,2,5]oxadiazolo[3,4-b]pyrazine-5,6-diamine;
(iii) N5,N6-bis(2,5-dichlorophenyl)-[1,2,5]oxadiazolo[3,4-b]pyrazine-5,6-diamine;
(jjj) N5-(3,4-dichlorophenyl)-N6-(piperidin-4-yl)-[1,2,5]oxadiazolo[3,4-b]pyrazine-5,6-diamine;
(kkk) N5-(4-chloro-3-methylphenyl)-N6-(3,4-dichlorophenyl)-[1,2,5]oxadiazolo[3,4-b]pyrazine-5,6-diamine;
(lll) 5,8-diisopropyl-5,6,7,8-tetrahydro-2-oxa-1,3,4,5,8,9-hexaaza-cyclopenta[b]naphthalene;
(mmm)6-(4-methylpiperazin-1-yl)-N—(naphthalen-1-yl)-[1,2,5]oxadiazolo[3,4-b]pyrazin-5-amine;
(nnn) 5-phenyl-5,6,7,8-tetrahydro-2-oxa-1,3,4,5,8,9-hexaaza-cyclopenta[b]naphthalene;
(ooo) 6-(N-methyl—N—phenylamino)-[1,2,5]oxadiazolo[3,4-b]pyrazin-5-ol;
(ppp) 5,8-diphenyl-5,6,7,8-tetrahydro-2-oxa-1,3,4,5,8,9-hexaaza-cyclopenta[b]naphthalene;
(qqq) tert-butyl2-(5-(p-tolylamino)-[1,2,5]oxadiazolo[3,4-b]pyrazin-6-ylamino) acetate;
(rrr) tert-butyl2-(5-(naphthalen-1-ylamino)-[1,2,5]oxadiazo lo [3,4-b]pyrazin-6-ylamino)acetate
(sss) N-(2-(5-hydroxy-[1,2,5]oxadiazolo[3,4-b]pyrazin-6-ylamino)ethyl)acetamide;
(ttt) N5,N6-bis(tert-butoxy-carbonylmethyl)-[1,2,5]oxadiazolo [3,4-b]pyrazine-5,6-diamine;
(uuu) N-(3,4-dichlorophenyl)-6-morpholino-[1,2,5]oxadiazolo [3,4-b]pyrazin-5-amine;
(vvv) 5,7-diphenyl-5,6,7,8-tetrahydro-2-oxa-1,3,4,5,8,9-hexaaza-cyclopenta[b]naphthalene;
(www) 1-(4-(5-(3,4-dichlorophenylamino)-[1,2,5]oxadiazolo[3,4-b]pyrazin-6-yl)piperazin-1-yl)ethanone;
(xxx) 5,6-dimorpholino-[1,2,5]oxadiazolo[3,4-b]pyrazine;
(yyy) 6-methoxy-N-(4-nitrophenyl)-[1,2,5]oxadiazolo[3,4-b]pyrazin-5-amine;
(zzz) 6-(naphthalen-1-ylamino)-[1,2,5]oxadiazolo[3,4-b]pyrazin-5-ol;
(aaaa) N5-(3,4-dichlorophenyl)-N6-methyl-[1,2,5]oxadiazo lo [3,4-b]pyrazine-5,6-diamine;
(bbbb) 2-[6-(1-tert-butoxycarbonyl-2-methyl-propylamino)-[1,2,5]oxadiazolo[3,4-b]pyrazin-5-ylamino]-3-methyl-butyric acid tert-butyl ester;
(cccc) (6R, 7S)-6,7-diphenyl-5,6,7,8-tetrahydro-2-oxa-1,3,4,5,8,9-hexaaza-cyclopenta[b]naphthalene;
(dddd) (6R, 7R)-6,7-diphenyl-5,6,7,8-tetrahydro-2-oxa-1,3,4,5,8,9-hexaaza-cyclopenta[b]naphthalene;
(eeee) (1,2,5]oxadiazolo[3,4-b]pyrazine-5,6-diamine;
(ffff) (S)-2-(5-((S)-1-(carboxy)-2-methylpropylamino)-[1,2,5]oxadiazolo[3,4-b]pyrazin-6-ylamino)-3-methylbutanoic acid;
(gggg) tert-butyl2-(5-(naphthalen-1-ylamino)-[1,2,5]oxadiazolo[3,4-b]pyrazin-6-ylamino) acetate;
(hhhh) (6S, 7S)-6,7-diphenyl-5,6,7,8-tetrahydro-2-oxa-1,3,4,5,8,9-hexaaza-cyclopenta[b]naphthalene;
(iiii) 6-morpholino-N-(naphthalen-1-yl)[1,2,5]oxadiazolo[3,4-b]pyrazin-5-amine;
(jjjj) 1,2-bis-(6-hydroxy-[1,2,5]oxadiazolo[3,4-b]pyrazin-5-ylamino)-ethane-1,2-diol;
(kkkk) N5,N6-dimethyl-N5,N6-diphenyl-[1,2,5]oxadiazolo[3,4-b]pyrazine-5,6-diamine;
(llll) 2-(5-(carboxymethylamino)-[1,2,5]oxadiazolo[3,4-b]pyrazin-6-ylamino) acetic acid;
(mmmm) 2-(5-(p-tolylamino)-[1,2,5]oxadiazolo[3,4-b]pyrazin-6-ylamino) acetic acid;
(nnnn) (6S, 7R)-6,7-diphenyl-5,6,7,8-tetrahydro-2-oxa-1,3,4,5,8,9-hexaaza-cyclopenta[b]naphthalene;
(oooo) 5,6-bis(4-methylpiperazin-1-yl)[1,2,5]oxadiazolo[3,4-b]pyrazine;
(pppp) 5,10-dihydro-2-oxa-1,3,4,5,10,11-hexaaza-cyclopenta[b]anthracene;
(qqqq) 9,15-dihydrodibenzo[f,h][1,2,5]oxadiazolo[3′,4′:5,6]pyrazino [2,3-b]quinoxaline;
(rrrr) 6,7-dicarboxy-5,6,7,8-tetrahydro-2-oxa-1,3,4,5,8,9-hexaaza-cyclopenta[b]naphthalene;
(ssss) N5,N6-bis(2-(dimethylamino)ethyl)-[1,2,5]oxadiazolo[3,4-b]pyrazine-5,6-diamine;
(tttt) 4-(5-(4-iodophenylamino)-[1,2,5]oxadiazolo[3,4-b]pyrazin-6-ylamino)-1,2-dihydro-2,3-dimethyl-1-phenylpyrazol-5-one;
(uuuu) N5,N5,N6,N6-tetramethyl-[1,2,5]oxadiazolo[3,4-b]pyrazine-5,6-diamine;
(vvvv) N5,N5-dimethyl-[1,2,5]oxadiazolo[3,4-b]pyrazine-5,6-diamine;
(wwww) N-(2-(5-(3,4-dichlorophenylamino)-[1,2,5]oxadiazolo[3,4-b]pyrazin-6-ylamino)-2-methylpropyl)-5-((4S ,6aR)-hexahydro-2-oxo-1H-thieno [3,4-d]imidazol-4-yl)pentanamide; and
(xxxx) 4-(5-(4-fluorophenylamino)-[1,2,5]oxadiazolo[3,4-b]pyrazin-6-ylamino)-1,2-dihydro-2,3-dimethyl-1-phenylpyrazol-5-one.

By “alkyl” and “lower alkyl” in the present invention, either alone or within other terms such as “alkylamino”, is meant straight or branched chain alkyl groups having 1-12 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is within a distinct range, it will be so indicated in the second “C” as, for example, “C₁-C₆” indicates a maximum of 6 carbons. The alkyl groups herein may be substituted in one or more substitutable positions with various groups. For example, such alkyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono (C₁-C₆)alkylamino (C₁-C₆)alkyl, di(C₁-C₆)alkylamino(C₁-C₆)alkyl or ═O.

By “alkoxy” and “lower alkoxy” in the present invention is meant straight or branched chain alkyl groups having 1-12 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy. The alkoxy groups herein may be substituted in one or more substitutable positions with various groups. For example, such alkoxy groups may be optionally substituted with C₁-C₆ alkyl, C₁-C₆ alkoxy, halogen, hydroxy, cyano, nitro, amino, mono (C₁-C₆)alkylamino , di(C₁-C₆)alkylamino, C₂-C₆alkenyl, C₂-C₆alkynyl, Cₗ-C₆ haloalkyl, C₁-C₆ haloalkoxy, amino (C₁-C₆)alkyl, mono (C₁-C₆)alkylamino (C₁-C₆)alkyl, di(C₁-C₆)alkylamino(C₁-C₆)alkyl or ═O.

The term “alkenyl” or “lower alkyenyl” embraces linear or branched radicals having at least one carbon-carbon double bond of two to twelve atoms. More preferred alkenyl radicals are those radicals having two to about four carbon atoms. Examples of alkenyl radicals include ethenyl, 2-propenyl, allyl, butenyl and 4-methylbutenyl. The terms “alkenyl” and “lower alkenyl”, embrace radicals having “cis” and “trans” orientations, or alternatively, “E” and “Z” orientations. The alkenyl groups herein may be alkenyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono (C₁-C₆)alkylamino (C₁-C₆)alkyl, di(C₁-C₆)alkylamino(C₁-C₆)alkyl or ═O.

The term “alkynyl” embraces linear or branched radicals having at least one carbon-carbon triple bond of two to twelve carbon atoms. More preferred alkynyl radicals are those radicals having two to about four carbon atoms. Examples of alkynyl radicals include ethynyl, 2-propynyl, and 4-methylbutynyl. The alkynyl groups herein may be substituted in one or more substitutable positions with various groups. For example, such alkynyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(Cᵢ-C₆)alkyl, mono (C₁-C₆)alkylamino (C₁-C₆)alkyl, di(C₁-C₆)alkylamino(C₁-C₆)alkyl or ═O.

The term “halo” or “halogen” means halogens such as fluorine, chlorine, bromine or iodine atoms.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), wherein such rings may be attached together in a pendent manner or may be fused. The term “aryl” embraces aromatic radicals such as phenyl, naphthyl, tetrahydronaphthyl, indane and biphenyl. More preferred aryl is phenyl. The aryl groups herein may be substituted in one or more substitutable positions with various groups. For example, such aryl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino (C₁-C₆)alkyl, mono (C₁-C₆)alkylamino (C₁-C₆)alkyl, di(C₁-C₆)alkylamino(C₁-C₆)alkyl or ═O.

By “heteroaryl” is meant one or more aromatic ring systems of 5-, 6-, or 7-membered rings which includes fused ring systems of 9-11 atoms containing at least one and up to four heteroatoms selected from nitrogen, oxygen, or sulfur. Examples include, but are not limited to, pyrrolyl, pyrrolinyl, imidazolyl, pyrazolyl, 2-pyridinyl, 3-pyridinyl, 4-pyridinyl, pyrimidinyl, pyrazinyl and pyridazinyl. The heteroaryl groups herein may be substituted in one or more substitutable positions with various groups. For example, such heteroaryl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono (C₁-C₆)alkylamino (C₁-C₆)alkyl, di(C₁-C₆)alkylamino(C₁-C₆)alkyl or ═O.

As used herein, the term “cycloalkyl” refers to saturated carbocyclic radicals having three to twelve carbon atoms. The cycloalkyl can be monocyclic, or a polycyclic fused system, and can optionally contain a double bond. Examples of such radicals include cyclopropyl, cyclobutyl, cyclopentyl, cyclohexyl and cycloheptyl. The cycloalkyl groups herein are unsubstituted or, as specified, substituted in one or more substitutable positions with various groups. For example, such cycloalkyl groups may be optionally substituted with C₁-C₆ alkyl, C₁-C₆ alkoxy, halogen, hydroxy, cyano, nitro, amino, oxo, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino (C₁-C₆)alkyl, mono (C₁-C₆)alkylamino (C₁-C₆)alkyl or di(C₁-C₆)alkylamino(C₁-C₆)alkyl.

By “heterocycle” or “heterocycloalkyl” is meant one or more carbocyclic ring systems which includes fused ring systems of 9-11 atoms containing at least one and up to four heteroatoms selected from nitrogen, oxygen, or sulfur. The heterocycle may optionally contain a double bond. Examples of heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide , oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein may be substituted in one or more substitutable positions with various groups. For example, such heterocycle groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino (C₁-C₆)alkyl or ═O.

Any term that includes two radicals such as, for example, “arylalkyl”, denotes the first radical, or aryl as in the example, attached to the concluding radical, or alkyl as in the example. The concluding radical is attached to the substituent in question.

Compounds of the present invention can possess, in general, one or more asymmetric carbon atoms and are thus capable of existing in the form of optical isomers as well as in the form of racemic or non-racemic mixtures thereof. Unless otherwise indicated, the compounds of the present invention, as depicted or named, may exist as the racemate, a single enantiomer, or any uneven (i.e. non 50/50) mixture of enantiomers. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column, such as, for example, a CHIRAL-AGP column, optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using optically active starting materials. These isomers may be in the form of a free acid, a free base, an ester or a salt.

Non-limiting examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

As used herein, the term “amount effective” means a dosage sufficient to produce a desired result. The desired result can be subjective or objective improvement in the recipient of the dosage; a decrease in tumor size, time to progression of disease, and/or survival; inhibiting an increase in tumor size; reducing or preventing metastases; and/or limiting or preventing recurrence of the tumor in a subject that has previously had a tumor.

In one embodiment, the methods of the invention can be used in combination with surgery on the subject, wherein surgery includes primary surgery for removing one or more tumors, secondary cytoreductive surgery, and palliative secondary surgery.

In a further embodiment, the methods of the invention further comprise treating the subject with chemotherapy and/or radiation therapy. One benefit of such a method if that use of the compounds permits a reduction in the chemotherapy and/or radiation dosage necessary to inhibit tumor growth and/or metastasis. As used herein, “radiotherapy” includes but is not limited to the use of radio-labeled compounds targeting tumor cells.

Any reduction in chemotherapeutic or radiation dosage benefits the patient by resulting in fewer and decreased side effects relative to standard chemotherapy and/or radiation therapy treatment. In this embodiment, the one or more compounds may be administered prior to, at the time of, or shortly after a given round of treatment with chemotherapeutic and/or radiation therapy. In a preferred embodiment, the one or more compounds is administered prior to or simultaneously with a given round of chemotherapy and/or radiation therapy. In a most preferred embodiment, the one or more compounds is administered prior to or simultaneously with each round of chemotherapy and/or radiation therapy. The exact timing of compound administration will be determined by an attending physician based on a number of factors, but the compound is generally administered between 24 hours before a given round of chemotherapy and/or radiation therapy and simultaneously with a given round of chemotherapy and/or radiation therapy.

The methods of the invention are appropriate for use with chemotherapy using one or more cytotoxic agent (ie: chemotherapeutic), including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al., Cancer Chemotherapeutic Agents: Handbook of Clinical Data (1978), hereby incorporated by reference. When administered as a combination, the therapeutic agents can be formulated as separate compositions that are given at the same time or different times, or the therapeutic agents can be given as a single composition.

The methods of the invention are also particularly suitable for those patients in need of repeated or high doses of chemotherapy and/or radiation therapy. The actual compound dosage range for administration is based on a variety of factors, including the age, weight, sex, medical condition of the individual, the severity of the condition, and the route of administration. Thus, the dosage regimen may vary widely, but can be determined by a physician using standard methods. An effective amount of the one or more compounds that can be employed ranges generally between 0.01 μg/kg body weight and 10 mg/kg body weight, preferably ranging between 0.05 μg/kg and 5 mg/kg body weight, more preferably between 1 μg/kg and 5 mg/kg body weight, and even more preferably between about 10 μg/kg and 5 mg/kg body weight.

The compounds may be made up in a solid form (including granules, powders or suppositories) or in a liquid form (e.g., solutions, suspensions, or emulsions). The compounds of the invention may be applied in a variety of solutions and may be subjected to conventional pharmaceutical operations such as sterilization and/or may contain conventional adjuvants, such as preservatives, stabilizers, wetting agents, emulsifiers, buffers etc. The compounds of the invention may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrasternal, intratendinous, intraspinal, intracranial, intrathoracic, infusion techniques, intracavity, or intraperitoneally.

In yet further aspects, the invention provides an article of manufacture comprising packaging material and the above pharmaceutical compositions.

The instant invention may be embodied in other forms or carried out in other ways without departing from the spirit or essential characteristics thereof. The present disclosure and enumerated examples are therefore to be considered as in all respects illustrative and not restrictive, and all equivalency are intended to be embraced therein. One of ordinary skill in the art would be able to recognize equivalent embodiments of the instant invention, and be able to practice such embodiments using the teaching of the instant disclosure and only routine experimentation.

### EXAMPLES

### Compound Synthesis

The compounds of the invention can be synthesized by procedures known in the art, for example, see Fernandez, E. et al., Tetradron Letters, Vol. 43, 4741-45 (2002) and Starchenkov, I. B., et al., Chemistry of Heterocyclic Comnpounds, Vol. 33, No. 10, 1219-33 (1997). A representative example is given in Example 1.

### Example 1

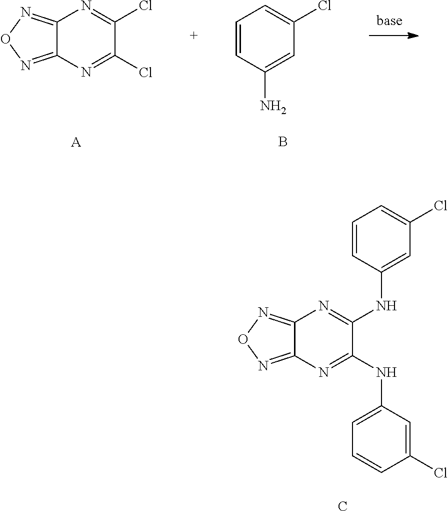

N,N′-Bis-(3-chloro-phenyl)-[1,2,5]oxadiazolo[3,4-b]pyrazine-5,6-diamine
To a dry roundbottom flask was added A and ethyl acetate (80 ml). To this stirred solution under a nitrogen atmosphere at −600 C. was added dropwise a solution of B and N,N′-diethylaniline in ethyl acetate (20 ml). A purple solution was rapidly formed during addition. The cooling bath was removed and the reaction mixture was allowed to warm to RT overnight. The reaction mixture was partitioned against water, washed with brine and dried over MgSO₄. The solvent was evaporated to dryness to give a solid. This solid was washed with hexanes to remove most of the impurities. An attempt to dissolve the solid in dichloromethane (200 ml) was made but an off-white solid didn't dissolve and was filtered off. When the dichloromethane was reduced to 100 ml a second batch of off-white solids was filtered off. Finally the volume was reduced to 50 ml to give a third batch of off-white solids. All batches were pure according to 1H-NMR. (NMR nr.: 9000013152094). The yield of the pure compound was 2469 mg (6.62 mmol, 74%). ¹H NMR (DMSO-d₆)δ 10.07 (2H, s); 8.00 (2H, s); 7.78 (1H, d); 7.52 (2H, dd); 7.31 (2H, d); NMR ID-nr.: H_RR_{—}2034416; Melting point: 237.8-238.50 C.

### Example 2

### Tissue Processing

Excess tissue specimens obtained from organs and tissues such as lung and testicle were obtained freshly at the time of surgery and samples were sent for pathological testing. For diagnosis and grading of tissue samples (ie: prior to processing), hematoxylin and eosin stained tissue sections were examined by a pathologist. If the diagnosis and grading of the tissue concurred with the determination made by the surgical pathologist that provided the tissue, then the tissue was used in the screen. If there was no agreement, then two additional pathologists served as referees. If no consensus was reached, then the tissue was discarded.

The remaining tissue was used to prepare cell suspensions. The tissue was initially treated enzymatically via standard methods until only undigested material remained. The digested cell suspension was filtered through one or more screens of between 40 micron and 100 micron porosity. The resulting cell suspension was further purified via isokinetic density centrifugation.

Additional normal cells were removed from the cell suspension by negative immunoselection with a combination of monoclonal antibodies linked to magnetic beads (Dynal) that were used according to the manufacturers' instructions. The remaining cells were placed into appropriate medium, frozen down in 1.0 mL aliquots, and stored until use.

### Example 3

### General Screen/Bioassay Procedures

After tissue processing, the relative purity of the resulting cell suspension was determined by cytological examination after pap staining Only those cell preparations greater than 80% tumor cells were used for testing of candidate compounds. If there was any doubt about the percentage of tumor cells in the cell preparation, additional pathologists served as referees to make a determination.

Cell preparations that passed histological and cytological examination for diagnosis, grading, and cell purity were thawed at 37° C. and resuspended in tissue culture medium designed to maintain the cells during the incubation period. The live and dead cells were counted and the cells were diluted in culture medium to 1.0×10³ live cells/test well for tumor cells and 3.3×10³ live cells/test well for normal cells.

The cells were added to microtiter plates and incubated at 37° C. overnight with 10 μM of the candidate compounds that were added at 1/10th the volume of the cell suspension. Alamar Blue (Accumed International, Westlake Ohio) was then added to the cells at 1/10 the volume of the well, and the cells were further incubated at 370 C. for various times. Alamar Blue dye measures cellular re-dox reactions (ie: cellular respiration) whereby a spectral shift occurs upon reduction of the dye. (Excitation 530 nm; emission 590 nm)

The kinetics of cellular re-dox reactions were subsequently measured at various times, for example at 3 hours, 3 days, and 5 days post-dye addition. These measurements, in comparison with control cells (untreated with compound) and media controls (test wells without cells) provide the percent inhibition of cellular mitochondrial respiration as a result of candidate compound treatment, as well as IC₅₀ determinations.

The Alamar Blue data were subsequently confirmed by microscopic observation, and by the use of calcein AM (Molecular Probes, Eugene Oreg.), a cell permeant esterase substrate that measures both esterase activity and cell membrane activity. If the cell is alive, the dye is converted into a fluorogenic substrate by intracellular esterases and is retained by the cell (excitation 485 nm; emission 530 nm). If the cells are dead, the calcein AM rapidly leaks from the cells and is not converted into a fluorogenic substrate. Thus, the assay is useful for cytotoxicity testing.

### Example 3

### Anti-Tumor Screen

In a blinded fashion, approximately 340,000 samples (representing approximately five million compounds) were tested at a rate of 1,000-4,000 compounds per run set against soft tissue sarcoma tumors, while approximately 10,000 of the compounds were also tested against colon and lung tumors. The anti-tumor screen utilized was composed of four tiers as follows. In Screen 1, patient tumor cells were tested in singles, with candidate samples. Samples that showed at least 80% inhibition (compared to cell and media controls) and/or at least two standard deviations from the mean of the plate samples were advanced. In the second test (Screen 2), the compounds were re-tested, in replicate, by serial dilution on patient tumors and the potency (IC₅₀) was determined. Samples that demonstrated nM potency for purified compounds, or microgram/ml potency for natural product extracts, were advanced to the third test (Screen 3). Samples were tested in Screen 3, in a dose-responsive manner, on both patients' tumor cells and normal cells. Samples that were greater than or equal to three times greater potency on tumor cells than normal cells were advanced to fourth test (Screen 4). Compounds were tested in Screen 4 were tested against a wide range of patients' tumor cells of differing anatomical locations and histological origins (sarcomas, melanomas, neuroblastomas, mesotheliomas, and carcinomas including lung, renal, ovarian, liver, bladder, and pancreatic) and normal cells from different anatomical locations (lung, renal, liver, spleen, ovary, peripheral blood mononuclear cells and heart). Those compounds that exhibit greater than, or equal to, three-fold greater potency for the majority of tumor cells rather than normal cells, were advanced for further evaluation and testing.

Using the screen disclosed above, a large number of compounds were analyzed for their anti-tumor activity (T-Sarcoma; T-Ovary: Ovarian carcinoma; and T-Lung: Non-small cell lung carcinoma). Compounds according to the present invention with activity against at least one tumor type tested are presented in Appendix A. IC50 values are reported for the designated tumor type, according to the methods disclosed in the specification.

The IC50 values are in micromolar concentrations and the acronyms used in the Tables are as follows:

T=Tumor

NT=Not tested

NT*=The compound showed activity at one, or more concentrations, but an IC50 was not determined; these compounds are considered “active”

NA=No activity observed

These data clearly show that the compounds of the invention can be used as an anti-tumor agent against a variety of tumor types.

What is claimed is:

1. A method for treating a tumor, comprising administering to a subject with a tumor an amount effective to treat the tumor of a compound of the formula: 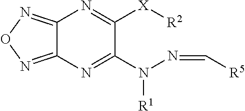
or a pharmaceutically acceptable derivative thereof,
wherein
X is O, S, or NR;
R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN;
R and R¹ independently are hydrogen or lower alkyl;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
R⁷ and R⁸ are independently hydrogen, lower alkyl, aryl, heteroaryl, arylalkyl, heteroarylalkyl, —C(O)R¹⁰ or —C(O)OR′;
R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
R′ is hydrogen or lower alkyl.

2. The method of claim 1 wherein
R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

3. The method of claim 1 wherein
R and R¹ are hydrogen.

4. The method of claim 1 wherein
R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

5. The method of claim 1 wherein X is —NR—.

6. The method of claim 1 wherein the compound has the formula: 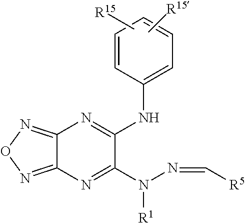
wherein
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′, —C(O)NR⁷R⁸, —C(O)R¹⁰ and —CN.

7. The method of claim 6 wherein
R¹ is hydrogen.

8. The method of claim 6 wherein
R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl, heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

9. The method of claim 6 wherein
R¹⁵ and R¹⁵ are independently hydrogen, trifluoromethyl, nitro, lower alkoxy, halo or lower alkyl.

10. The method of claim 1 wherein the compound has the formula: 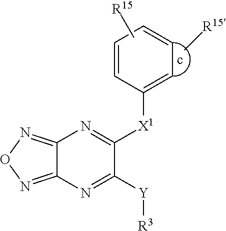
wherein
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁶ and —CN.

11. The method of claim 10 wherein
R¹ is hydrogen.

12. The method of claim 10 wherein
R⁵ is aryl or heteroaryl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁶, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁶, nitro and —CN.

13. The method of claim 10 wherein
R¹⁵ are independently hydrogen, trifluoromethyl, nitro, lower alkoxy, halo or lower alkyl.

14. The method of claim 1 wherein the compound has the formula: 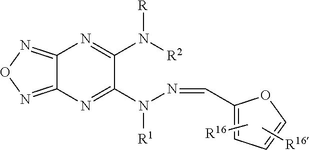
wherein
R¹⁶ and R^{16′} independently are hydrogen, lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

15. The method of claim 14 wherein
R and R¹ are hydrogen.

16. The method of claim 14 wherein
R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

17. The method of claim 14 wherein
R^{16′} independently are hydrogen, lower alkyl, lower alkoxy, nitro, halo, or aryl optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

18. The method of claim 1 wherein the compound has the formula: 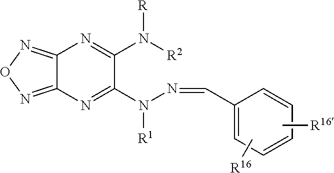
wherein
R¹⁶ and R^{16′} independently are hydrogen, lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

19. The method of claim 18 wherein
wherein
R and R¹ are hydrogen.

20. The method of claim 18 wherein
R² is aryl or cycloalkyl, each of which may be optionally substituted with one or two groups independently selected from lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN.

21. The method of claim 18 wherein
R¹⁶ and R^{16′} independently are hydrogen, lower alkyl, lower alkoxy, nitro, halo, or aryl optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN.

22. The method of claim 1 wherein the compound is selected from the group consisting of
6-(2-((furan-2-yl)methylene)hydrazinyl)-N-(2-iodophenyl)-[1,2,5]oxadiazolo[3,4-b]pyrazin-5-amine;
6-(2-((5-methylfuran-2-yl)methylene)hydrazinyl)-N-(4-iodophenyl)-[1,2,5]oxadiazolo[3,4-b]pyrazin-5-amine;
6-(2-45-(3-nitrophenyl)furan-2-yl)methylene)hydrazinyl)-N-(4-chloro-2-iodophenyl) [1,2,5]oxadiazolo[3,4-b]pyrazin-5-amine;
6-(2-((furan-2-yl)methylene)hydrazinyl)-N-m-tolyl-[1,2,5]oxadiazolo[3,4-b]pyrazin-5-amine;
6-(2-((2-hydroxy-5-nitro-phenyl-1-yl)methylene)hydrazinyl)-N-(4-chloro-phenyl) [1,2,5]oxadiazolo[3,4-b]pyrazin-5-amine;
6-(2-((2-hydroxy-5-bromo-phenyl)methylene)hydrazinyl)-N-(4-chloro-phenyl)-[1,2,5]oxadiazolo[3,4-b]pyrazin-5-amine;
6-(2-45-(2-chloro-5-nitrophenyl)furan-2-yl)methylene)hydrazinyl)-N—cyclohexyl-[1,2,5]oxadiazolo[3,4-b]pyrazin-5-amine;
6-(2-((furan-2-yl)methylene)hydrazinyl)-N—cyclohexyl-[1,2,5]oxadiazolo[3,4-b]pyrazin-5-amine;
6-(2-45-(2-methoxy-4-nitrophenyl)furan-2-yl)methylene)hydrazinyl)-N-(4-chlorophenyl) [1,2,5]oxadiazolo[3,4-b]pyrazin-5-amine;
64(E)-245-(4-bromophenyl)furan-2-yl)methylene)hydrazinyl)-N—p-tolyl-[1,2,5]oxadiazolo [3,4-b]pyrazin-5-amine; and
6-(2-((2-hydroxy-5-nitro-phenyl)methylene)hydrazinyl)-N-(2,3-dimethyl-phenyl)-[1,2,5]oxadiazolo[3,4-b]pyrazin-5-amine.

23. A method for treating a tumor, comprising administering to a subject with a tumor an amount effective to treat the tumor of a compound of the formula: 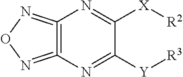
or a pharmaceutically acceptable derivative thereof wherein
X is O, S, or NR;
Y is O, S, or NR¹;
R and R¹ are independently hydrogen or lower alkyl;
R² is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, aryl, heteroaryl, cycloalkyl, heterocycloalkyl, —C(O)OR′, —C(O)NR⁷R⁸, —C(O)R¹⁰ and —CN, or
R² is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰,or
R² is heteroarylalkyl, or
R and R², when Y is —NR¹, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R³ is hydrogen, or
R³ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, —C(O)OR′, —C(O)NR⁷R⁸, —C(O)R¹⁰ and —CN, or
R³ is —N═CH—R⁵, or
R³ is lower alkyl optionally substituted with one, two or three groups independently selected from halo, hydroxyl, trifluoromethyl, —OR⁶, —NR⁷R⁸, nitro, —C(O)OR′, —C(O)NR⁷R⁸, —CN, aryl, heteroaryl, cycloalkyl, heterocycloalkyl and —C(O)R¹⁰,or
R¹ and R³, when Y is —NR^{I}, together with the nitrogen atom to which they are attached, form a 5-7 membered heterocyclic ring optionally substituted with one or two groups selected from lower alkyl, —C(O)R¹⁰, —C(O)OR′, —C(O)NR⁷R⁸, oxo, aryl, halo, hydroxyl, trifluoromethyl, —NR⁷R⁸ and lower alkoxy, or
R² and R³, together with the heteroatom that they are attached, form a 5-20 membered heterocyclic ring optionally substituted with one or two groups selected from lower alkyl, —C(O)R¹⁰, —C(O)OR′, —OC(O)R′, —C(O)NR⁷R⁸, oxo, aryl, halo, hydroxyl, trifluoromethyl, —NR⁷R⁸ and lower alkoxy;
R⁵ is aryl, heteroaryl, cycloalkyl or heterocycloalkyl, each of which may be optionally substituted with one, two, three or four groups independently selected from lower alkyl, lower alkenyl, lower alkynyl, halo, hydroxyl, trifluoromethyl, lower alkoxy, (C₁-C₆)alkoxy(C₁-C₆)alkyl, —NR⁷R⁸, nitro, oxo, aryl, heteroaryl, cycloalkyl, heterocycloalkyl, —OCF₃, —SO₂NR⁷R⁸, —C(O)OR′, —C(O)NR⁷R⁸, —C(O)R¹⁰, —CN, or
aryl heteroaryl, cycloalkyl or heterocycloalkyl, each of which may be optionally substituted with one or two groups independently selected from lower alkyl, lower alkoxy, hydroxyl, trifluoromethyl, halo, —C(O)OR′, —C(O)NR⁷R⁸, —C(O)R¹⁰, nitro and —CN;
R⁶ is lower alkyl optionally substituted with one or two groups selected from hydroxyl and lower alkoxy,
R⁷ and R⁸ are independently hydrogen, lower alkyl, —C(O)R¹⁰ or —C(O)OR′, or
R⁷ and R⁸ are independently aryl, heteroaryl, arylalkyl, heteroarylalkyl, each of which may be optionally substituted with one or two groups selected from lower alkyl, lower alkoxy, oxo, hydroxyl or amino;
R¹⁰ is selected from hydrogen, lower alkyl, aryl, heteroaryl, cycloalkyl, heterocycloalkyl, arylalkyl, heteroarylalkyl, cycloalkylalkyl and heterocyclolalkylalkyl; and,
R′ is hydrogen or lower alkyl; and pharmaceutically acceptable derivatives thereof

24. The method of claim 23 wherein the compound has the formula: 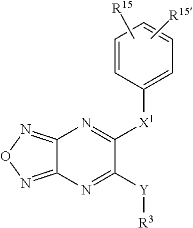
wherein
X¹ is O or —NR—; and
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′, —C(O)NR⁷R⁸, —C(O)R¹⁰ and —CN.

25. The method of claim 23 wherein the compound has the formula: 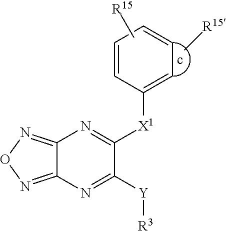
wherein
X¹ is O or —NR—;
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁰ and —CN, and
the c-ring, together with the phenyl ring to which it is attached, is a 9-11 membered aryl, heteroaryl, cycloalkyl or heterocyclyl group.

26. The method of claim 23 wherein the compound has the formula: 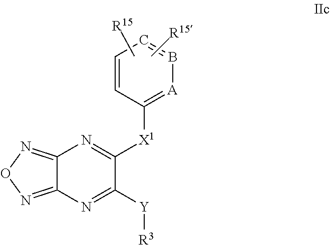
wherein
X¹ is O or —NR—;
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁰ and —CN, and
one of A, B and C is N.

27. The method of claim 23 wherein the compound has the formula: 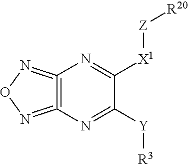
wherein
X¹ is O or —NR—;
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁰ and —CN; and
Z is lower alkyl optionally substituted with one or two groups selected from hydroxyl, lower alkoxy or halo, and R²⁰ is nothing or —C(O)OR′, —NC(O)R′, —NR⁷R⁸, aryl or heteroaryl.

28. The method of claim 23 wherein the compound has the formula: 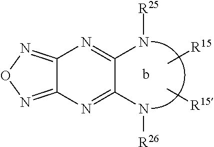
wherein
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁶ and —CN;
R²⁵ and R²⁶ are independently hydrogen, aryl or lower alkyl; and
the b-ring is a 5-20 membered heterocyclic ring.

29. The method of claim 23 wherein the compound has the formula: 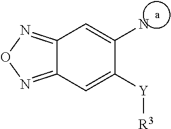
wherein
the a-ring, which includes the nitrogen atom, is a 5-7 membered heterocyclic ring optionally substituted with one or two groups that, independently, are R¹⁵ or R^{15′}; and
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁶ and —CN.

30. The method of claim 23 wherein the compound has the formula:

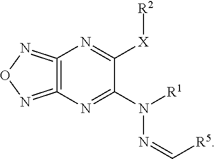

31. The method of claim 23 wherein the compound has the formula: 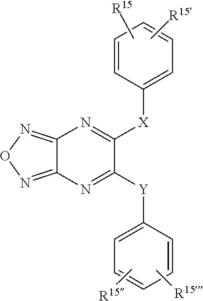
wherein
R¹⁵, R^{15′}, R^{15″} and R^{15′″} are independently selected from hydrogen, lower alkyl, lower alkenyl, lower alkynyl, halo, hydroxyl, trifluoromethyl, lower alkoxy, —(C₁-C₆) alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁰ and —CN.

32. The method of claim 23 wherein the compound has the formula: 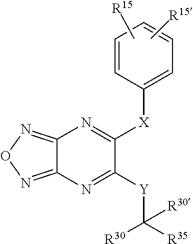
wherein
R¹⁵ and R^{15′} are independently selected from hydrogen, lower alkyl, lower alkenyl, lower alkynyl, halo, hydroxyl, trifluoromethyl, lower alkoxy, —(C₁-C₆)alkoxy(C₁-C₆)alkyl, —NR⁷R⁸, nitro, aryl, heteroaryl, cycloalkyl, heterocycloalkyl, —C(O)OR′, —OC(O)R′,—C(O)NR⁷R⁸, —C(O)R¹⁰ and —CN; and
R³⁰ and R^{30′} are independently hydrogen, arylalkyl or lower alkyl, and R³⁵ is hydrogen, lower alkyl, lower alkoxy, —C(O)OR′, —C(O)R¹⁰, —NR⁷R⁸, aryl, heteroaryl, cycloalkyl, or heterocycloalkyl.

33. The method of claim 23 wherein the compound is selected from the group consisting of:
N5,N6-bis(3-chlorophenyl)-[1,2,5]oxadiazolo[3,4-b]pyrazine-5,6-diamine;
N5-(3,4-dichlorophenyl)-N6-(4-methoxyphenyl)-[1,2,5]oxadiazolo[3,4-b]pyrazine-5,6-diamine;
N5,N6-bis(3-trifluoromethyl)-[1,2,5]oxadiazolo[3,4-b]pyrazine-5,6-diamine;
N5-butyl-N6-(3,4-dichlorophenyl)-[1,2,5]oxadiazolo[3,4-b]pyrazine-5,6-diamine;
N5-(3,4-dichlorophenyl)-N6-(2-chlorophenyl)-[1,2,5]oxadiazolo[3,4-b]pyrazine-5,6-diamine;
N5,N6-bis(4-chlorophenyl)-[1,2,5]oxadiazolo[3,4-b]pyrazine-5,6-diamine;
N5-(3,4-dichlorophenyl)-N6-(3-methoxyphenyl)-[1,2,5]oxadiazolo[3,4-b]pyrazine-5,6-diamine;
N5,N6-bis(4-chloro-3-methylphenyl)-[1,2,5]oxadiazolo[3,4-b]pyrazine-5,6-diamine;
(S)-tertbutyl-2-(5-(4-chloro-3-methylphenylamino)-[1,2,5]oxadiazolo[3,4-b]pyrazin-6-ylamino)-3-methylbutanoate ;
N5-(4-chlorophenyl)-N6-(2-chlorophenyl)[1,2,5]oxadiazolo[3,4-b]pyrazine-5,6-diamine;
N5,N6-bis(3,4-dichlorophenyl)-[1,2,5]oxadiazolo[3,4-b]pyrazine-5,6-diamine;
tert-butyl 2-(5-(3,4-dichlorophenylamino)-[1,2,5]oxadiazolo[3,4-b]pyrazin-6-ylamino)acetate;
N5-(4-methoxyphenyl)-N6-(3-bromophenyl)-[1,2,5]oxadiazolo[3,4-b]pyrazine-5,6-diamine;
N5-(3,4-dichlorophenyl)-N6-((furan-2-yl)methyl)-[1,2,5]oxadiazolo[3,4-b]pyrazine-5,6-diamine;
N5,N6-bis(4-trifluoromethylphenyl)-[1,2,5]oxadiazolo[3,4-b]pyrazine-5,6-diamine;
N5,N6-bis(4-methyl-2-nitrophenyl)-[1,2,5]oxadiazolo[3,4-b]pyrazine-5,6-diamine;
tert-butyl2-(5-(3,4-dichlorophenylamino)-[1,2,5]oxadiazolo[3,4-b]pyrazin-6-ylamino)-2-methylpropylcarb amate;
6-(3,4-dichlorophenoxy)-N—(naphthalen-1-yl)-[1,2,5]oxadiazolo[3,4-b]pyrazin-5-amine;
N5-(3-trifluormethylphenyl)-N6-(3-methylphenyl)-[1,2,5]oxadiazolo[3,4-b]pyrazine-5,6-diamine;
(5)-methyl 2-(5-(3,4-dichlorophenylamino)-[1,2,5]oxadiazolo[3,4-b]pyrazin-6-ylamino)-3-phenylpropanoate;
N-(3-chloro-4-methylphenyl)-6-morpholino-[1,2,5]oxadiazolo[3,4-b]pyrazin-5-amine;
N5,N6-bis(4-nitrophenyl)-[1,2,5]oxadiazolo[3,4-b]pyrazine-5,6-diamine;
N5,N6-bis(3-methylphenyl)-[1,2,5]oxadiazolo[3,4-b]pyrazine-5,6-diamine;
N5,N6-bis(4-fluorophenyl)-[1,2,5]oxadiazolo[3,4-b]pyrazine-5,6-diamine;
ethyl 4-(5-((furan-2-yl)methylamino)-[1,2,5]oxadiazolo[3,4-b]pyrazin-6-ylamino)benzoate;
N6-(3,4-dichlorophenyl)-N5-(4-chlorophenyl)-N5-methyl-[1,2,5]oxadiazolo[3,4-b]pyrazine-5,6-diamine;
N5,N6-bis(3-chloro-4-methylphenyl)-[1,2,5]oxadiazolo[3,4-b]pyrazine-5,6-diamine;
N5-(3,4-dichlorophenyl)-N6-ethyl-N6-phenyl[1,2,5]oxadiazolo[3,4-b]pyrazine-5,6-diamine;
(S)-2-(5-(3,4-dichlorophenylamino)-[1,2,5]oxadiazolo[3,4-b]pyrazin-6-ylamino)-3-methyl-1-morpholinobutan-1-one;
N5-tert-butyl-N6-(3,4-dichlorophenyl)-[1,2,5]oxadiazolo[3,4-b]pyrazine-5,6-diamine;
N5-(3,4-dichlorophenyl)-N6-isobutyl-[1,2,5]oxadiazolo[3,4-b]pyrazine-5,6-diamine;
N5,N6-bis(3-methylphenyl)-[1,2,5]oxadiazolo[3,4-b]pyrazine-5,6-diamine;
N5-(benzo[d][1,3]dioxo1-5-yl)-N6-(benzo [d][1,3]dioxo1-6-yl)-[1,2,5]oxadiazolo[3,4-b]pyrazine-5,6-diamine;
(S)-tert-butyl2-(5-(p-tolylamino)-[1,2,5]oxadiazolo[3,4-b]pyrazin-6-ylamino)-3-methylbutanoate;
N5,N6-bis(phenyl)[1,2,5]oxadiazolo[3,4-b]pyrazine-5,6-diamine;
N5-((benzo[d][1,3]dioxol-5-yl)methyl)-N6-((benzo[d][1,3]dioxo1-6-yl)methyl)-[1,2,5]oxadiazolo[3,4-b]pyrazine-5,6-diamine;
N5-(3,4-dichlorophenyl)-N6-(2-methoxyethyl)-[1,2,5]oxadiazolo[3,4-b]pyrazine-5,6-diamine;
5,6-bis(3,4-dichlorophenoxy)-[1,2,5]oxadiazolo[3,4-b]pyrazine;
N5,N6-bis(4-methylphenyl)-[1,2,5]oxadiazolo[3,4-b]pyrazine-5,6-diamine;
4-(5-(3,4-dichlorophenylamino)-[1,2,5]oxadiazolo[3,4-b]pyrazin-6-ylamino)benzoic acid;
(5)-ethyl 2-(5-(3,4-dichlorophenylamino)-[1,2,5]oxadiazolo[3,4-b]pyrazin-6-ylamino)propanoate;
N5,N6-bis(3,4-dimethoxyphenyl)-[1,2,5]oxadiazolo[3,4-b]pyrazine-5,6-diamine;
methyl 2-(5-(3,4-dichlorophenylamino)-[1,2,5]oxadiazolo[3,4-b]pyrazin-6-ylamino)acetate;
N5,N6-bis(6-chloropyridin-3-yl)-[1,2,5]oxadiazolo[3,4-b]pyrazine-5,6-diamine;
N5-(2-(1H-imidazol-4-yl)ethyl)-N6-(3,4-dichlorophenyl)-[1,2,5]oxadiazolo[3,4-b]pyrazine-5,6-diamine;
N5-(5-(2-morpholinoethoxy)pyridin-2-yl)-N6-(naphthalen-1-yl)-[1,2,5]oxadiazolo[3,4-b]pyrazine-5,6-diamine;
N5-(1-amino-2-methylpropan-2-yl)-N6-(3,4-dichlorophenyl)-[1,2,5]oxadiazolo[3,4-b]pyrazine-5,6-diamine;
N5-(3,4-dichlorophenyl)-N6-(naphthalen-1-yl)-[1,2,5]oxadiazolo[3,4-b]pyrazine-5,6-diamine;
N5-(2-((pyridin-4-yl)methylamino)ethyl)-N6-(3,4-dichlorophenyl)-[1,2,5]oxadiazolo[3,4-b]pyrazine-5,6-diamine;
N5-(3,4-dichlorophenyl)-N6-(2-(dimethylamino)ethyl)[1,2,5]oxadiazolo[3,4-b]pyrazine-5,6-diamine;
3-(3-(5-(3,4-dichlorophenylamino)-[1,2,5]oxadiazolo[3,4-b]pyrazin-6-ylamino)propoxy)propane-1,2-diol;
N5-(3,4-dichlorophenyl)-N6-((piperidin-4-yl)methyl)[1,2,5]oxadiazolo[3,4-b]pyrazine-5,6-diamine;
N5,N6-bis(4-methoxyphenyl)-[1,2,5]oxadiazolo[3,4-b]pyrazine-5,6-diamine;
N5-isopropyl-N6-phenyl-[1,2,5]oxadiazolo[3,4-b]pyrazine-5,6-diamine;
4-(5-(2,3-dimethylphenylamino)-[1,2,5]oxadiazolo[3,4-b]pyrazin-6-ylamino)-1,2-dihydro-2,3-dimethyl-1-phenylpyrazol-5-one;
N5,N6-bis((furan-2-yl)methyl)-[1,2,5]oxadiazolo[3,4-b]pyrazine-5,6-diamine;
6-(3,4-dichlorophenoxy)-N-(3,4-dichlorophenyl)-[1,2,5]oxadiazolo[3,4-b]pyrazin-5-amine;
6-(3,4-dichlorophenylamino)-[1,2,5]oxadiazolo[3,4-b]pyrazin-5-ol;
4-(5-(3,4-dichlorophenylamino)-[1,2,5]oxadiazolo[3,4-b]pyrazin-6-ylamino)-1,2-dihydro-2,3-dimethyl-1-phenylpyrazol-5-one;
N5,N6-bis(3-bromophenyl)-[1,2,5]oxadiazolo[3,4-b]pyrazine-5,6-diamine;
N5,N6-bis(2,5-dichlorophenyl)-[1,2,5]oxadiazolo[3,4-b]pyrazine-5,6-diamine;
N5-(3,4-dichlorophenyl)-N6-(piperidin-4-yl)-[1,2,5]oxadiazolo[3,4-b]pyrazine-5,6-diamine;
N5-(4-chloro-3-methylphenyl)-N6-(3,4-dichlorophenyl)-[1,2,5]oxadiazolo[3,4-b]pyrazine-5,6-diamine;
5, 8-diisopropyl-5,6,7,8-tetrahydro-2-oxa-1,3,4,5,8,9-hexaaza-cyclopenta[b]naphthalene;
6-(4-methylpiperazin-1-yl)-N—(naphthalen-1-yl)-[1,2,5]oxadiazolo[3,4-b]pyrazin-5-amine;
5-phenyl-5,6,7,8-tetrahydro-2-oxa-1,3,4,5,8,9-hexaaza-cyclopenta[b]naphthalene;
6-(N-methyl—N—phenylamino)-[1,2,5]oxadiazolo[3,4-b]pyrazin-5-ol;
5,8-diphenyl-5,6,7,8-tetrahydro-2-oxa-1,3,4,5,8,9-hexaaza-cyclopenta[b]naphthalene;
tert-butyl 2-(5-(ptolylamino)[1,2,5]oxadiazolo[3,4-b]pyrazin-6-ylamino)acetate;
tert-butyl 2-(5-(naphthalen-1-ylamino)-[1,2,5]oxadiazolo[3,4-b]pyrazin-6-ylamino)acetate N-(2-(5-hydroxy-[1,2,5]oxadiazolo[3,4-b]pyrazin-6-ylamino)ethyl)acetamide;
N5,N6-bis(tert-butoxy-carbonylmethyl)[1,2,5]oxadiazolo[3,4-b]pyrazine-5,6-diamine;
N-(3,4-dichlorophenyl)-6-morpholino-[1,2,5]oxadiazolo[3,4-b]pyrazin-5-amine;
5,7-diphenyl-5,6,7,8-tetrahydro-2-oxa-1,3,4,5,8,9-hexaaza-cyclopenta[b]naphthalene;
1-(4-(5-(3,4-dichlorophenylamino)-[1,2,5]oxadiazolo[3,4-b]pyrazin-6-yl)piperazin-1-yl)ethanone;
5,6-dimorpholino-[1,2,5]oxadiazolo[3,4-b]pyrazine;
6-methoxy-N-(4-nitrophenyl)-[1,2,5]oxadiazolo[3,4-b]pyrazin-5-amine;
6-(naphthalen-1-ylamino)-[1,2,5]oxadiazolo[3,4-b]pyrazin-5-ol;
N5-(3,4-dichlorophenyl)-N6-methyl-[1,2,5]oxadiazolo[3,4-b]pyrazine-5,6-diamine;
2-[6-(1-tert-butoxycarbonyl-2-methyl-propylamino)-[1,2,5]oxadiazolo[3,4-b]pyrazin-5-ylamino]-3-methyl-butyric acid tert-butyl ester;
(6R, 7S)-6,7-diphenyl-5,6,7,8-tetrahydro-2-oxa-1,3,4,5,8,9-hexaaza-cyclopenta[b]naphthalene;
(6R, 7R)-6,7-diphenyl-5,6,7,8-tetrahydro-2-oxa-1,3,4,5,8,9-hexaaza-cyclopenta[b]naphthalene;
(1,2,5]oxadiazolo[3,4-b]pyrazine-5,6-diamine;
(S)-2-(5-((S)-1-(carboxy)-2-methylpropylamino)-[1,2,5]oxadiazolo[3,4-b]pyrazin-6-ylamino)-3-methylbutanoic acid;
tert-butyl 2-(5-(naphthalen-1-ylamino)-[1,2,5]oxadiazolo[3,4-b]pyrazin-6-ylamino)acetate;
(6S, 7S)-6,7-diphenyl-5,6,7, 8-tetrahydro-2-oxa-1,3,4,5,8,9-hexaaza-cyclopenta[b]naphthalene;
6-morpholino—N—(naphthalen-1-yl)[1,2,5]oxadiazolo[3,4-b]pyrazin-5-amine;
1,2-bis-(6-hydroxy-[1,2,5]oxadiazolo[3,4-b]pyrazin-5-ylamino)-ethane-1,2-diol;
N5,N6-dimethyl-N5,N6-diphenyl-[1,2,5]oxadiazolo[3,4-b]pyrazine-5,6-diamine;
2-(5-(carboxymethylamino)-[1 ,2,5]oxadiazolo[3,4-b]pyrazin-6-ylamino)acetic acid;
2-(5-(p-tolylamino)-[1,2,5]oxadiazolo[3,4-b]pyrazin-6-ylamino)acetic acid;
(6S, 7R)-6,7-diphenyl-5,6,7,8-tetrahydro-2-oxa-1,3,4,5,8,9-hexaaza-cyclopenta[b]naphthalene;
5,6-bis(4-methylpiperazin-1-yl)[1,2,5]oxadiazolo[3,4-b]pyrazine;
5,10-dihydro-2-oxa-1,3,4,5,10,11-hexaaza-cyclopenta[b]anthracene;
9,15-dihydrodibenzo [f, h][1,2,5]oxadiazolo[3′,4′:5,6]pyrazino [2,3-b]quinoxaline;
6,7-dicarboxy-5,6,7,8-tetrahydro-2-oxa-1,3,4,5,8,9-hexaaza-cyclopenta[b]naphthalene;
N5,N6-bis(2-(dimethylamino)ethyl)-[1,2,5]oxadiazolo[3,4-b]pyrazine-5,6-diamine;
4-(5-(4-iodophenylamino)-[1,2,5]oxadiazolo[3,4-b]pyrazin-6-ylamino)-1,2-dihydro-2,3-dimethyl-1-phenylpyrazol-5-one;
N5,N5,N6,N6-tetramethyl-[1,2,5]oxadiazolo[3,4-b]pyrazine-5,6-diamine;
N5,N5-dimethyl-[1,2,5]oxadiazolo[3,4-b]pyrazine-5,6-diamine;
N-(2-(5-(3,4-dichlorophenylamino)-[1,2,5]oxadiazolo[3,4-b]pyrazin-6-ylamino)-2-methylpropyl)-5-((4S,6aR)-hexahydro-2-oxo-1H-thieno[3,4-d]imidazol-4-yl)pentanamide; and
4-(5-(4-fluorophenylamino)-[1,2,5]oxadiazolo[3,4-b]pyrazin-6-ylamino)-1,2-dihydro-2,3-dimethyl-1-phenylpyrazol-5-one.

34. The method of claim 1 wherein the tumor is selected from the group consisting of sarcoma, melanoma, neuroblastoma, carcinoma, and mesothelioma.

35. The method of claim 23 wherein the tumor is selected from the group consisting of sarcoma, melanoma, neuroblastoma, carcinoma, and mesothelioma.
